(19) United States
(12) Patent Application Publication
(10) Pub. No.: US 2022/0232831 A1
(43) Pub. Date: Jul. 28, 2022

(54) NOVEL NON-CODING HETEROCYCLIC AMINO ACIDS (NCHAA) AND THEIR USE AS HERBICIDES

(71) Applicant: Fortephest Ltd., Ness Ziona (IL)

(72) Inventors: Alex Kozak, Ness Ziona (IL); Israel Shapiro, Rehovot (IL)

(73) Assignee: Fortephest Ltd., Ness Ziona (IL)

(21) Appl. No.: 17/618,330

(22) PCT Filed: Jun. 10, 2020
(86) PCT No.: PCT/IL2020/050642

§ 371 Date: Dec. 10, 2021

Related U.S. Application Data
(60) Provisional application No. 62/860,045, filed on Jun. 11, 2019.

(51) Int. Cl. A01N 43/78; A01N 43/10; A01N 43/54; A01N 43/40; A01N 43/82; A01N 43/08; A01N 43/76; A01N 43/60; A01N 43/56; A01N 43/36; A01N 43/653; A01N 43/50; A01N 43/647; A01N 43/713; A01P 13/00; A01P 21/00

(52) U.S. Cl. CPC A01N 43/78; A01P 21/00; A01N 43/54; A01N 43/40; A01N 43/82; A01N 43/08; A01N 43/76; A01N 43/60; A01N 43/56; A01N 43/36; A01N 43/653; A01N 43/50; A01N 43/647; A01N 43/713; A01P 13/00; A01N 43/10

(57) ABSTRACT

The invention relates to a novel substituted amino acids, agricultural compositions comprising the novel substituted amino acids, and their use for controlling undesired plant growth alone or in combination with crop protection agents such as pesticides or plant growth regulators.

### FIELD OF THE INVENTION

The invention relates to novel chemical compounds having herbicidal activity, process for their manufacture and their use in crop protection.

### BACKGROUND OF THE INVENTION

Fidelity of protein synthesis is critically important as it ensures functional activity of cell proteins. However, some non-coded amino acids, despite not being encoded by the genetic code, are nevertheless found in proteins. Error rates of translation in vivo have been estimated to be on the order of 10₋₃-10₋₄. However, high synthesis rates impose constraints on accuracy that lead to an increased the number of erroneous cellular proteins. As such, nascent proteins in rapidly growing organs such as plants meristems in germinating and early developing periods are more prone to misincorporation amino acids.

Incorporation of non-coded amino acids in the peptide chain can make proteins dysfunctional or toxic. It was further suggested that herbicidal activity of natural non-coded amino acid meta-tyrosine and some of its derivatives relates to its incorporation in plant proteins Bertin et al., 2007b; WO2017141253.

New compounds effective for controlling the growth of undesired vegetation are in constant demand. In the most common situation, such compounds are sought to selectively control the growth of weeds in useful crops such as cotton, rice, corn, wheat and soybeans, to name a few. Unchecked weed growth in such crops can cause significant losses, reducing profit to the farmer and increasing costs to the consumer. In other situations, herbicides are desired which will control all plant growth. Examples of areas in which complete control of all vegetation is desired are areas around railroad tracks, storage tanks and industrial storage areas. Identifying new compounds having herbicidal activity which are more effective, less costly and environmentally safe remains long and unmet need.

### SUMMARY OF THE INVENTION

It is a principal object of the present invention to provide novel, non-coded amino acids as potent herbicides.

The invention provides a compound having the structure

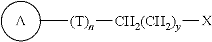

or a salt thereof, wherein:

A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and, wherein when the heteroatoms of the ring are N or S, each independently is optionally substituted with R₃, R₄, R₆ and R₇;

T is selected from the group consisting of, sulfonyl, ammonium, alkylamine; arylamine, phosphonium, nitro, cyano, dihalomethyl, acyl, formyl, haloformyls, carboxyl, alkoxycarbonyl, and aminocarbonyl;

n is 0 or 1;

y is 0 to 8;

X is

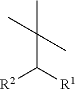

wherein R₁ is selected from the group consisting of carboxyl group, carbamoyl group, ester with saturated or non-saturated alcohols with straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide.

The invention further provides an agricultural composition comprising the compound having the structure

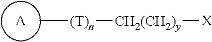

or a salt thereof, wherein:

A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and, wherein, when one or more heteroatoms of the ring is N or S, each independently is optionally substituted with R₃, R₄, R₆ and R₇;

T is selected from the group consisting of, sulfonyl, ammonium, alkylamine; arylamine, phosphonium, nitro, cyano, dihalomethyl, acyl, formyl, haloformyls, carboxyl, alkoxycarbonyl, and aminocarbonyl;

n is 0 or 1;

y is 0 to 8;

X is

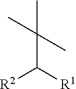

wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide, and at least one agriculturally acceptable carrier.

The invention provides a method of controlling undesired plant growth, comprising applying to the locus of the undesired plant growth a herbicidally effective amount of a compound having the structure

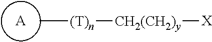

or a salt thereof, wherein:

A is five, six or nine-membered ring comprising one or more heteroatoms; wherein each of the heteroatoms is independently selected from the group consisting of N, S, and O; and wherein one or more of the carbon atoms of the ring are optionally substituted with R₃, R₄, R₆ and R₇; and wherein each of R₃, R₄, R₆ or R₇ is independently selected from an electron withdrawing groups (EWG) or an electron donating groups (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;

T is selected from the group consisting of sulfonyl, ammonium, alkylamines and arylamines, phosphonium, nitro, cyano, dihalomethyl, acyl, formyl, haloformyls, carboxyl, alkoxycarbonyl, and aminocarbonyl;

n is 0 or 1;

y is 0 to 8;

X is

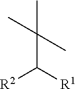

wherein R₁ is selected from the group consisting of carboxyl group, carbamoyl group, ester with saturated or non-saturated aliphatic straight, branched, cyclic chain, or aromatic chain, ethylene glycol, polyethylene glycol, propylene glycol, polypropylene glycol, carboxamide substituted with straight, branched, cyclic aliphatic or aromatic chain, ethylene glycol, polyethylene glycol, propylene glycol, polypropylene glycol, sulfonamide; and phosphoramide; and wherein R₂ is selected from the group consisting of optionally substituted primary amine, secondary amine, tertiary amine, carboxamides, sulfonamide, and phosphoramide.

The invention further provides a method of controlling undesired plant growth comprising applying to the locus of the undesired plant growth:
a. A first herbicide having the structure

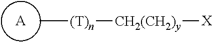

or a salt thereof, wherein:
A is five, six or nine-membered ring comprising one or more heteroatoms; wherein each of the heteroatoms is independently selected from the group consisting of N, S, and O; and wherein one or more of the carbon atoms of the ring are optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;
T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;
n is 0 or 1;
y is 0 to 8;
X is

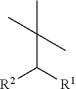

wherein R₁ is selected from the group consisting of carboxyl group, carbamoyl group, ester with saturated or non-saturated aliphatic straight, branched or cyclic chain, ethylene glycol, polyethylene glycol, propylene glycol, polypropylene glycol; and wherein R₂ is selected from the group consisting of optionally substituted primary amine; secondary amine; tertiary amine; and an amide derivative of carboxylic or inorganic acid; and
b. A second herbicide, to effectively control the undesired plant growth.

### DETAILED DESCRIPTION OF THE INVENTION

The present invention is now described more fully hereinafter with reference to the accompanying examples, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

In on embodiment, the invention provides a compound having the structure

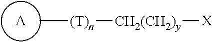

or a salt thereof, wherein:

A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;

T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;

n is 0 or 1;

y is 0 to 8;

X is

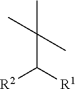

wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide. In one embodiment, A is selected from the group consisting of pyrrole, pyrazole, imidazole, triazole, thiophene, dithiole, trithiole, thiazole, dithiazole, trithiazolidine, thiadiazole, dithiadiazole, thiatriazole, oxazole, furan, oxathiole, pyridine, pyridazine, pyrimidine, pyrazine, triazine, thiazine, dithiazine, thiadiazine, 2H-1,3,4,5-thiatriazine, dithiadiazine, pyrrolopyridine, pyrrolopyrimidine, pyrrolotriazine, thiopyranopyrrole, pyrrolothiazine, pyrrolodithiazine, and thienopyridine. In yet another embodiment, A is selected from the group consisting of thiophene, 2H-1,3-dithiole, 3H-1,2-dithiole, 1H-pyrrole, 2H-1,2,3-triazole, 1H-1,2,4-triazole, 1,3-thiazole, 1,2,5-thiadiazole, 1,2,4-thiadiazole, 1,3-oxazole, furan, and pyridine. In one embodiment, the EWG is selected from the group consisting of triflyl (trifluoromethanesulfonyl), trihalide, cyano, sulfonate, nitro, ammonium, quaternary amine, aldehyde, ketone, carboxylic acid, ester, amide, and halide. As used herein, the term “electron withdrawing group” (EWG) refers, without limitation, to an atom or a group that draws electron density from neighboring atoms or aromatic ring, usually by resonance or inductive effects. As used herein, the term “electron donating group” (EDG) refers, without limitation, to an atom or a group that donates electron density to neighboring atoms or aromatic ring, usually by resonance or inductive effects. In one embodiment, the EDG is selected from the group consisting of phenoxide, tertiary amine, secondary amine, primary amine, ether, alkyl, phenyl and vinyl. In yet further embodiment, R₂ is primary, secondary and tertiary amine substituted with a group selected from hydrogen, an aliphatic compound with straight or branched chain, an aliphatic compound with non-aromatic ring, straight or branched-chain alcohol, alcohol with non-aromatic ring, phosphoric acid, phosphonic acid, terminal carbon of phosphonate, ethylene glycol, polyethylene glycol, propylene glycol and polypropylene glycol. In another embodiment, R₂ is selected from the group consisting of carboxamide with saturated or non-saturated straight, branched or cyclic chain, sulfonamide, and phosphoramide.

In one embodiment the invention provides a compound having the structure:

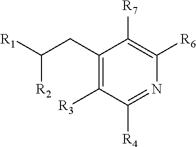

wherein R₁ is COOH, COOCH₃, COOC₂H₅, COOC₃H₇; R₂ is NH₂; R₃ and R₇ are independently selected from H, CF₃, CCl₃, Cl, and F; and R₄ and R₆ are independently selected from H, Cl, and Br.

In another embodiment, the invention provides a compound having the structure:

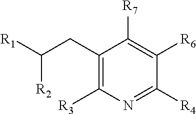

wherein R₁ is selected from COOH, COOCH₃, COOC₂H₅, and COOC₃H₇; R₂ is NH₂; R₃ and R₄ are independently selected from H, Br and Cl; and R₆ and R₇ are independently selected from H, Cl, NO₂, CF₃, and CCl₃.

In yet another embodiment, the invention provides a compound having the structure:

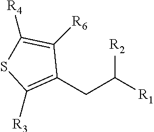

wherein R₁ is selected from COOH, COOCH₃, COOC₂H₅, COOC₄H₉, CONHCH₂COOH, and COO(CH₂)₃CH₃; R₂ is selected from NH₂, NHCOCH₃, NHCO, and NH(CH₂)₂O(CH₂)₂)OH; R₃ and R₄ are independently selected from H, Cl, Br, CN, CHF₂ CF₃, CCl₃; and R₆ is H or CF₃. In one embodiment, the invention provides a compound having the structure:

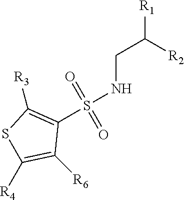

wherein R₁ is selected from COOH, COOCH₃, COOC₂H₅, COOC₅H₁₁, and COO(CH₂)₂(CH₂)₂CH₃; R₂ is NH₂; R₃ and R₄ are independently selected from H, Br, and Cl; and R₆ is selected from H, F, Cl, Br, CF₃, CCl₃.

In one embodiment, the invention provides a compound having the structure:

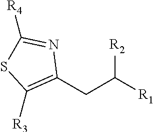

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂HB; R₃ and R₄ are each independently selected from H, Br, Cl, and NH₂.

In another embodiment, the invention provides a compound having the structure:

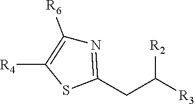

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂HB; R₄ and R₆ are each independently selected from H, Br, Cl, CF₃, CHF₂, and CCl₃.

In one embodiment, the invention provides a compound having the structure:

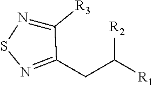

wherein R₁ is COOH; R₂ is NH₂; and R₆ is H. In yet further embodiment, the invention provides a compound having the structure:

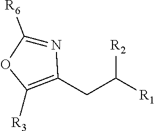

wherein R₁ is OH; R₂ is NH₂; and R₃ and R₆ are both H.

In another embodiment, the invention provides a compound having the structure:

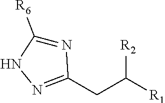

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂.

In one embodiment, the invention provides a compound having the structure:

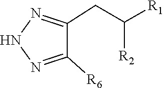

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂.

In one embodiment, the invention provides a compound having the structure:

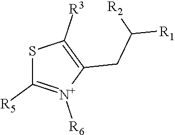

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₆ is selected from CH₃, OH and NH₂. In one embodiment, the invention provides a compound having the structure:

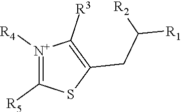

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₄ is selected from CH₃, OH and NH₂.

In one embodiment, provided an agricultural composition comprising a compound having the structure

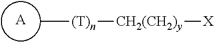

or a salt thereof, wherein:

A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;

T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;

n is 0 or 1;

y is 0 to 8;

X is

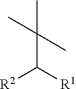

wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide; and at least one agriculturally acceptable carrier. In one embodiment, A is selected from the group consisting of pyrrole, pyrazole, imidazole, triazole, thiophene, dithiole, trithiole, thiazole, dithiazole, trithiazolidine, thiadiazole, dithiadiazole, thiatriazole, oxazole, furan, oxathiole, pyridine, pyridazine, pyrimidine, pyrazine, triazine, thiazine, dithiazine, thiadiazine, 2H-1,3,4,5-thiatriazine, dithiadiazine, pyrrolopyridine, pyrrolopyrimidine, pyrrolotriazine, thiopyranopyrrole, pyrrolothiazine, pyrrolodithiazine, and thienopyridine. In yet another embodiment, A is selected from the group consisting of thiophene, 2H-1,3-dithiole, 3H-1,2-dithiole, 1H-pyrrole, 2H-1,2,3-triazole, 1H-1,2,4-triazole, 1,3-thiazole, 1,2,5-thiadiazole, 1,2,4-thiadiazole, 1,3-oxazole, furan, and pyridine. In one embodiment, the EWG is selected from the group consisting of triflyl (trifluoromethanesulfonyl), trihalide, cyano, sulfonate, nitro, ammonium, quaternary amine, aldehyde, ketone, carboxylic acid, ester, amide, and halide. In one embodiment, the EDG is selected from the group consisting of phenoxide, tertiary amine, secondary amine, primary amine, ether, alkyl, phenyl and vinyl. In yet further embodiment, R₂ is primary, secondary and tertiary amine substituted with a group selected from hydrogen, an aliphatic compound with straight or branched chain, an aliphatic compound with non-aromatic ring, straight or branched-chain alcohol, alcohol with non-aromatic ring, phosphoric acid, phosphonic acid, terminal carbon of phosphonate, ethylene glycol, polyethylene glycol, propylene glycol and polypropylene glycol. In another embodiment, R₂ is selected from the group consisting of carboxamide with saturated or non-saturated straight, branched or cyclic chain, sulfonamide, and phosphoramide.

In one embodiment the composition comprises a compound having the structure:

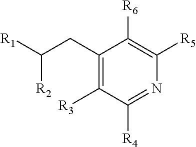

wherein R₁ is COOH, COOCH₃, COOC₂H₅, COOC₃H₇; R₂ is NH₂; R₃ and R₇ are independently selected from H, CF₃, CCl₃, Cl, and F; and R₄ and R₆ are independently selected from H, Cl, and Br. In another embodiment, the composition comprises a compound having the structure:

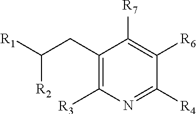

wherein R₁ is selected from COOH, COOCH₃, COOC₂H₅, and COOC₃H₇; R₂ is NH₂; R₃ and R₄ are independently selected from H, Br and Cl; and R₆ and R₇ are independently selected from H, Cl, NO₂, CF₃, and CCl₃. In yet another embodiment, the composition comprises a compound having the structure:

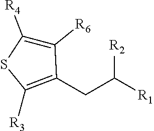

wherein R₁ is selected from COOH, COOCH₃, COOC₂H₅, COOC₄H₉, CONHCH₂COOH, and COO(CH₂)₃CH₃; R₂ is selected from NH₂, NHCOCH₃, NHCO, and NH(CH₂)₂O(CH₂)₂)OH; R₃ and R₄ are independently selected from H, Cl, Br, CN, CHF2 CF₃, CCl₃; and R₆ is H or CF₃. In one embodiment, the composition comprises a compound having the structure:

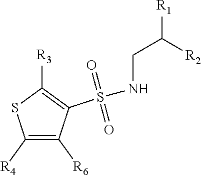

wherein R₁ is selected from COOH, COOCH₃, COOC₂H₅, COOC₅H₁₁, and COO(CH₂)₂(CH₂)₂CH₃; R₂ is NH₂; R₃ and R₄ are independently selected from H, Br, and Cl; and R₆ is selected from H, F, Cl, Br, CF₃, CCl₃. In one embodiment, the composition comprises a compound having the structure:

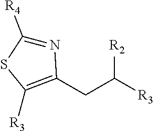

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₄ are each independently selected from H, Br, Cl, and NH₂. In another embodiment, the composition comprises a compound having the structure:

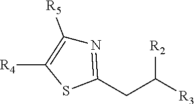

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₄ and R₆ are each independently selected from H, Br, Cl, CF₃, CHF₂, and CCl₃. In one embodiment, the composition comprises a compound having the structure:

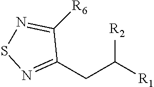

wherein R₁ is COOH; R₂ is NH₂; and R₆ is H. In yet further embodiment, the composition comprises a compound having the structure:

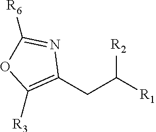

wherein R₁ is OH; R₂ is NH₂; and R₃ and R₆ are both H. In another embodiment, the composition comprises a compound having the structure:

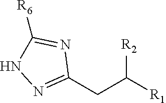

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂. In one embodiment, the composition comprises a compound having the structure:

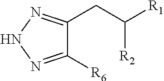

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂. In one embodiment, the composition comprises a compound having the structure:

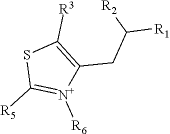

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₆ is selected from CH₃, OH and NH₂. In one embodiment, the composition comprises a compound having the structure:

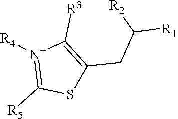

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₄ is selected from CH₃, OH and NH₂.

In one embodiment, the agricultural composition further comprises at least one crop protection agent. In another embodiment, the at least one crop protection agent is selected from the group consisting of fungicide, insecticide, herbicide, and plant growth regulator. In yet another embodiment, the crop protection agent is herbicide. In yet another embodiment, the crop protection agent is plant growth regulator. In one embodiment, the at least one crop protection agent is selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop(P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof.

In one embodiment of the invention provided a method of controlling undesired plant growth comprising applying to the locus of said undesired plant growth the agricultural composition comprising a compound having the structure

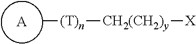

or a salt thereof, wherein:

A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;

T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;

n is 0 or 1;

y is 0 to 8;

X is

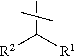

wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide; and at least one agriculturally acceptable carrier. In one embodiment, A is selected from the group consisting of pyrrole, pyrazole, imidazole, triazole, thiophene, dithiole, trithiole, thiazole, dithiazole, trithiazolidine, thiadiazole, dithiadiazole, thiatriazole, oxazole, furan, oxathiole, pyridine, pyridazine, pyrimidine, pyrazine, triazine, thiazine, dithiazine, thiadiazine, 2H-1,3,4,5-thiatriazine, dithiadiazine, pyrrolopyridine, pyrrolopyrimidine, pyrrolotriazine, thiopyranopyrrole, pyrrolothiazine, pyrrolodithiazine, and thienopyridine. In yet another embodiment, A is selected from the group consisting of thiophene, 2H-1,3-dithiole, 3H-1,2-dithiole, 1H-pyrrole, 2H-1,2,3-triazole, 1H-1,2,4-triazole, 1,3-thiazole, 1,2,5-thiadiazole, 1,2,4-thiadiazole, 1,3-oxazole, furan, and pyridine. In one embodiment, the EWG is selected from the group consisting of triflyl (trifluoromethanesulfonyl), trihalide, cyano, sulfonate, nitro, ammonium, quaternary amine, aldehyde, ketone, carboxylic acid, ester, amide, and halide. In one embodiment, the EDG is selected from the group consisting of phenoxide, tertiary amine, secondary amine, primary amine, ether, alkyl, phenyl and vinyl. In yet further embodiment, R₂ is selected from the group consisting of primary, secondary and tertiary amine substituted with a group selected from hydrogen, an aliphatic compound with straight or branched chain, an aliphatic compound with non-aromatic ring, straight or branched-chain alcohol, alcohol with non-aromatic ring, carboxylic acid, phosphoric acid, phosphonic acid, terminal carbon of phosphonate, ethylene glycol, polyethylene glycol, propylene glycol and polypropylene glycol. In another embodiment, R₂ is selected from the group consisting of carboxamide with saturated or non-saturated straight, branched or cyclic chain, sulfonamide, and phosphoramide.

In one embodiment the composition comprises a compound having the structure:

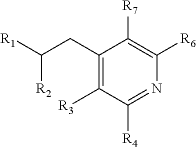

wherein R₁ is COOH, COOCH₃, COOC₂H₅, COOC₃H₇; R₂ is NH₂; R₃ and R₇ are independently selected from H, CF₃, CCl₃, Cl, and F; and R₄ and R₆ are independently selected from H, Cl, and Br.

In another embodiment, the composition comprises a compound having the structure:

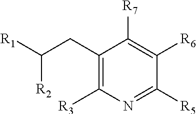

wherein R₁ is selected from COOH, COOCH₃, COOC₂H₅, and COOC₃H₇; R₂ is NH₂; R₃ and R₄ are independently selected from H, Br and Cl; and R₆ and R₇ are independently selected from H, Cl, NO₂, CF₃, and CCl₃.

In yet another embodiment, the composition comprises a compound having the structure:

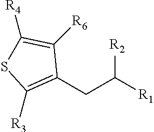

wherein R₁ is selected from COOH, COOCH₃, COOC₂H₅, COOC₄H₉, CONHCH₂COOH, and COO(CH₂)₃CH₃; R₂ is selected from NH₂, NHCOCH₃, NHCO, and NH(CH₂)₂O(CH₂)₂)OH; R₃ and R₄ are independently selected from H, Cl, Br, CN, CHF2 CF₃, CCl₃; and R₆ is H or CF₃. In one embodiment, the composition comprises a compound having the structure:

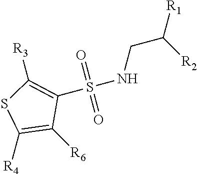

wherein R₁ is selected from COOH, COOCH₃, COOC₂H₅, COOC₅H₁₁, and COO(CH₂)₂(CH₂)₂CH₃; R₂ is NH₂; R₃ and R₄ are independently selected from H, Br, and Cl; and R₆ is selected from H, F, Cl, Br, CF₃, CCl₃.

In one embodiment, the composition comprises a compound having the structure:

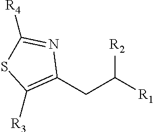

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₄ are each independently selected from H, Br, Cl, and NH₂.

In another embodiment, the composition comprises a compound having the structure:

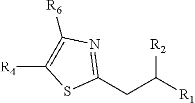

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₄ and R₆ are each independently selected from H, Br, Cl, CF₃, CHF₂, and CCl₃.

In one embodiment, the composition comprises a compound having the structure:

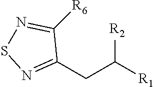

wherein R₁ is COOH; R₂ is NH₂; and R₆ is H.

In yet further embodiment, the composition comprises a compound having the structure:

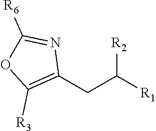

wherein R₁ is OH; R₂ is NH₂; and R₃ and R₆ are both H.

In another embodiment, the composition comprises a compound having the structure:

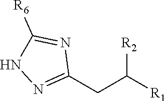

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂. In one embodiment, the composition comprises a compound having the structure:

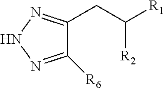

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂. In one embodiment, the composition comprises a compound having the structure:

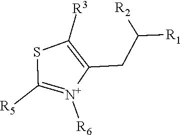

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₆ is selected from CH₃, OH and NH₂. In one embodiment, the composition comprises a compound having the structure:

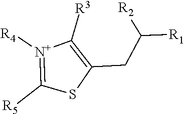

wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₄ is selected from CH₃, OH and NH₂.

In one embodiment, the method further comprises applying to the locus of the undesired plant growth at least one crop protection agent. In one embodiment, the crop protection agent is selected from the group consisting of herbicide, fungicide, insecticide and plant growth regulator. In one embodiment, the crop protection agent is herbicide. In one embodiment, the crop protection agent is amino acid synthesis inhibitor herbicide. In another embodiment, the amino acid synthesis inhibitor herbicide is selected form the group consisting of sulfonylurea herbicide, imidazolinone herbicide, sulfonamide herbicide and amino acid derivatives, or a combination thereof. In one embodiment, the amino acid synthesis inhibitor herbicide is selected form the group consisting of imazamox, imazapic, imazethapyr, imazaquin, imazapyr and imazamethabenz, or a combination thereof. In one embodiment, the amino acid synthesis inhibitor herbicide is selected form the group consisting of Chlorimuron, Primisulfuron, Thifensulfuron, Triasulfuron, Nicosulfuron, Metsulfuron, Tribenuron, Rimsulfuron and Triflusulfuron or a combination thereof. In another embodiment, the amino acid synthesis inhibitor herbicide is glyphosate. In yet another embodiment the crop protection agent is a plant growth regulator. In one embodiment, the plant growth regulator is selected from the group consisting of dicamba, 2,4-D, clopyralid and fluroxypyr.

In one embodiment the invention provides a method of controlling undesired plant growth comprising applying to the locus of the undesired plant growth:
a. A first herbicide having the structure

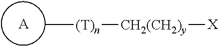

or a salt thereof, wherein:
A is five, six or nine-membered ring comprising one or more heteroatoms; wherein each of the heteroatoms is independently selected from the group consisting of N, S, and O; and wherein one or more of the carbon atoms of the ring are optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;
T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;
n is 0 or 1;
y is 0 to 8;
X is

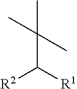

wherein R₁ is selected from the group consisting of carboxyl group, carbamoyl group, ester with saturated or non-saturated aliphatic straight, branched or cyclic chain, ethylene glycol, polyethylene glycol, propylene glycol, polypropylene glycol; and wherein R₂ is selected from the group consisting of optionally substituted primary amine; secondary amine; tertiary amine; and an amide derivative of carboxylic or inorganic acid; and
b. A second herbicide, to effectively control the undesired plant growth.

In one embodiment, the second herbicide is amino acid synthesis inhibitor herbicide. In one embodiment, the second herbicide is selected from the group consisting of sulfonylurea herbicide, imidazolinone herbicide, sulfonamide herbicide, and amino acid derivative. In another embodiment, the second herbicide is selected from the group consisting of imazamox, imazapic, imazethapyr, imazaquin, imazapyr, imazamethabenz, Chlorimuron, Primisulfuron, Thifensulfuron, Triasulfuron, Nicosulfuron, Metsulfuron, Tribenuron, Rimsulfuron, Triflusulfuron, and Glyphosate. In one embodiment, the second herbicide is selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop(P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, and ethametsulfuron-methyl. In one embodiment, the method of controlling undesired plant growth further comprises applying a third herbicide or a plant growth regulator.

In one embodiment, the invention provides a composition for controlling undesired plant growth comprising a mixture of:
a. A compound having the structure

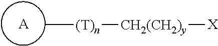

or a salt thereof, wherein:
A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and, wherein, when one or more heteroatoms of the ring is N or S, each independently is optionally substituted with R₃, R₄, R₆ and R₇;
T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;
n is 0 or 1;
y is 0 to 8;
X is

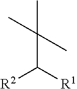

wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide;
b. at least one herbicide selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop(P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof; and
c. at least one agriculturally acceptable carrier.

In one embodiment, A is selected from the group consisting of pyrrole, pyrazole, imidazole, triazole, thiophene, dithiole, trithiole, thiazole, dithiazole, trithiazolidine, thiadiazole, dithiadiazole, thiatriazole, oxazole, furan, oxathiole, pyridine, pyridazine, pyrimidine, pyrazine, triazine, thiazine, dithiazine, thiadiazine, 2H-1,3,4,5-thiatriazine, dithiadiazine, pyrrolopyridine, pyrrolopyrimidine, pyrrolotriazine, thiopyranopyrrole, pyrrolothiazine, pyrrolodithiazine, and thienopyridine. In yet another embodiment, A is selected from the group consisting of thiophene, 2H-1,3-dithiole, 3H-1,2-dithiole, 1H-pyrrole, 2H-1,2,3-triazole, 1H-1,2,4-triazole, 1,3-thiazole, 1,2,5-thiadiazole, 1,2,4-thiadiazole, 1,3-oxazole, furan, and pyridine. In one embodiment, the EWG is selected from the group consisting of triflyl (trifluoromethanesulfonyl), trihalide, cyano, sulfonate, nitro, ammonium, quaternary amine, aldehyde, ketone, carboxylic acid, ester, amide, and halide. In one embodiment, the EDG is selected from the group consisting of phenoxide, tertiary amine, secondary amine, primary amine, ether, alkyl, phenyl and vinyl. In yet further embodiment, R₂ is primary, secondary and tertiary amine substituted with a group selected from hydrogen, an aliphatic compound with straight or branched chain, an aliphatic compound with non-aromatic ring, straight or branched-chain alcohol, alcohol with non-aromatic ring, phosphoric acid, phosphonic acid, terminal carbon of phosphonate, ethylene glycol, polyethylene glycol, propylene glycol and polypropylene glycol. In another embodiment, R₂ is selected from the group consisting of carboxamide with saturated or non-saturated straight, branched or cyclic chain, sulfonamide, and phosphoramide.

According to some embodiments, the invention provides a compound or a salt thereof, selected from the group consisting of: 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate.

According to some embodiments, the invention provides an agricultural composition comprising the compound or a salt thereof, selected from the group consisting of: 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate and at least one agriculturally acceptable carrier.

According to some embodiments, the invention provides a method of controlling undesired plant growth comprising applying to the locus of the undesired plant growth a compound or a salt thereof, selected from the group consisting of: 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate.

According to some embodiments, the invention provides a composition for controlling undesired plant growth comprising a mixture of: a) a compound selected from the group consisting of: 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate; b) a second herbicide; and, c) at least one agriculturally acceptable carrier.

According to some embodiments, the invention provides a mixture comprising: a) a compound selected from the group consisting of 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate; and, b) a second herbicide.

According to some embodiments, the invention provides a mixture comprising: a) a compound selected from the group consisting of 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate; and b) a plant growth regulator.

### EXAMPLES

In the examples below, if an abbreviation is not defined above, it has its generally accepted meaning. Further, all temperatures are in degrees Celsius (unless otherwise indicated). The following methods were used to prepare the compounds set forth below as indicated.

### Example 1: Synthesis of ethyl 2-acetamido-3-(2,5-dichloro-1,3-thiazol-4-yl)propanoate (Code 567)

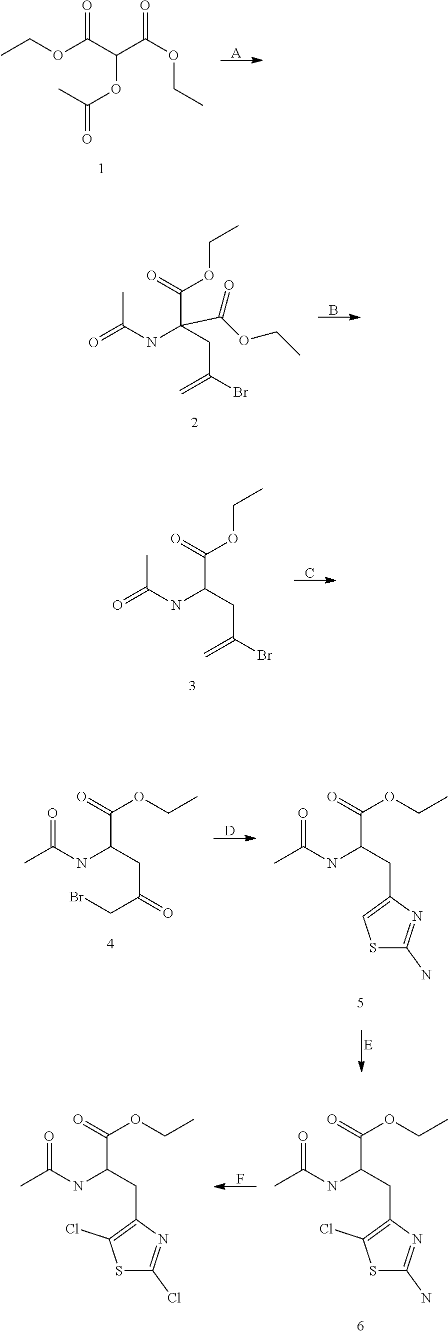

Step A: To a stirred mixture of NaH (44 g, 1.1 mol, 60% in silicone oil) in dry DMF (2.50 L) diethyl acetamidomalonate 1 (217 g, 1.0 mol) was added 2,3-dibromopropene (240 g, 1.2 mol). The reaction mixture was stirred at room temperature overnight to yield diethyl 2-acetamido-2-(2-bromoallyl) malonate. After completion of the reaction (confirmed by HNMR) the reaction mixture was used in the Step B.

Step B: To a solution of diethyl 2-acetamido-2-(2-bromoallyl)malonate obtained at the Step A, 336 g) water (45 mL, 2.5 mol) and LiBr (102 g, 1.2 mol) were subsequently added and the mixture was heated to reflux under argon for 6 h. The mixture was subsequently evaporated to remove DMF and quenched with EtOAc (1.5 L) and H₂O (1 L). The mixture was stirred for 10 min and extracted with EtOAc (3×0.5 L), the combined organic layers were washed with brine (3×0.5 L), dried over Na₂SO₄ anhydride) and concentrated in vacuo. Obtained oily residue (210 g, 93% purity) was used in the next step without purification.

Step C: To a cold (0° C.) stirred solution of 3 (210 g, 93% purity, 0.74 mol), under nitrogen, in a 2:1 mixture of acetonitrile/water (1.68 L) was added solid N-bromosuccinimide (193 g, 1.08 mole) in three portions over a period of 15 min. The resultant orange mixture was stirred at 0° C. for 1 h. The reaction mixture was treated at RT with 10% of aqueous sodium thiosulfate in ethyl acetate (1.5 L) and washed with water (4×300 mL) and brine (4×300 mL). Evaporation of the organic solvents results in the compound 4 (122 g, 0.43 mol) which was used in the next step without purification.

Step D: Compound 4 (122 g, 0.43 mol) and thiourea (33 g, 0.44 mol) were dissolved in EtOH (1.5 L) and stirred under reflux overnight. After the reaction was complete (confirmed by HNMR), the organic solvents were evaporated, and the crude residue was partitioned in EtOAc/H2O (1 L:1 L). The lower layer was carefully basified with 10% aq NaOH to pH=9 and extracted with CH₂Cl₂ (3×150 mL). Combined organic layers was washed with brine (2×100 mL), dried over Na₂SO₄ anhydride and evaporated, compound 5 (80 g, 0.28 mol)

Step E: Compound 5 (80 g, 0.28 mol) and N-chlorosuccinimide (56 g, 0.42 mol) were dissolved in acetonitrile (1.5 L). The mixture was stirred at ambient temperature for one hour. After the completion of the reaction, acetonitrile was evaporated under reduced pressure and the residue was partitioned in water/ethyl acetate (400 mL:400 mL). The layers were separated, and the aqueous layer was extracted with ethyl acetate (3×150 mL). The combined organic layers were washed with brine, dried over sodium sulfate, filtered and concentrated to give compound 6 (60 g, crude, 70% purity) which was used immediately in the next step without purification.

Step F: To a round bottomed flask was added crude compound 6 (60 g, crude, 70% purity), copper (II) chloride (38 g, 0.28 mol), and tert-butyl nitrite (35.6 mL, 0.3 mol) in CH₃CN (1 L). The mixture was heated to 40° C. for 2 hours, then the mixture was diluted with 1N HCl and extracted with EtOAc. The organic extract was washed with water (2×100 mL), brine (2×100 mL), dried over Na₂SO₄ anhydride, filtered and concentrated in vacuo to give the title compound, which was purified by CC to 95% purity (5.5 g, 0.018 mol).

Analytical data: ₁H NMR (400 MHz, CDCl₃) δ 6.42 (s, 1H), 4.99-4.75 (m, 1H), 4.17 (q, J=6.7 Hz, 2H), 3.30-2.99 (m, 2H), 2.00 (s, 1H), 1.24 (t, J=7.1 Hz, 3H); EδI: Calculated [M₊](C₁₀H₁₂O₃SN₂Cl₂) m/z: 311 AND 313. Found [M−e₋]₊, 311 and 312.9 [M+Cl₋]₋=346.8 and 348.7.

### Example 2: Synthesis of ethyl 2-amino-3-(2,5-dichlorothiophen-3-yl)propanoate (Code 569)

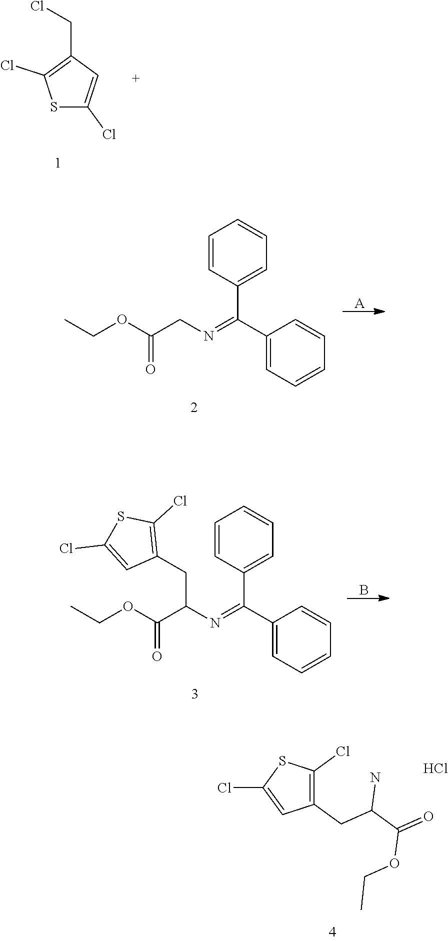

Step A. To a flame-dried flask containing t-BuOK (12.3 g, 109 mmol, 1.10 eq.) and anhydrous DMF (900 ml) was added ethyl 2-((diphenylmethylene)amino) acetate 2 (27.9 g, 104 mmol, 1.05 eq.) at 0° C. under argon. After 10 min, chloride 1 (20 g, 99 mmol, 1.0 eq.) was added. The resulting reaction mixture was stirred at 0° C. for 10 min and then at RT overnight. The reaction mixture was then slowly poured into water, extracted with DCM. The combined organic layers were dried over Na₂SO₄ anhydride, filtered and concentrated under reduced pressure to give compound 3 (29.0 g, 67.6%).

Step B. Compound 3 (29.0 g, 67 mmol) was treated with a mixture of EtOH/concentrated HCl (25:1 v/v) (700 ml). The resulting solution was stirred at room temperature for 2 h. After evaporation of the solvent the residue was rinsed with water, washed with hexane and concentrated under reduced pressure to yield the target product (16.9 g, 83.0%).

Analytical data: ₁H NMR (DMSO-d₆) δ_{H} (ppm): 1.12 (t, 3H, CH₃), 3.02-3.14 (dd, 2H, CH₂), 4.07-4.16 (m, 2H, CH₂), 4.20-4.26 (m, 1H, CH), 7.20 (s, 1H, HetAr.H), 8.67-8.78 (br.s, 3H, NH₂.HCl) EδI: Calculated [M₊](C₉H₁₁O₂NSCl₂) m/z: 268.0 and 270.0. Found [M−e₋]₊, 268.0 and 270.0.

### Example 3: Synthesis of 2-amino-3-(2,5-dibromothiophen-3-yl)propanoic Acid (Code 552)

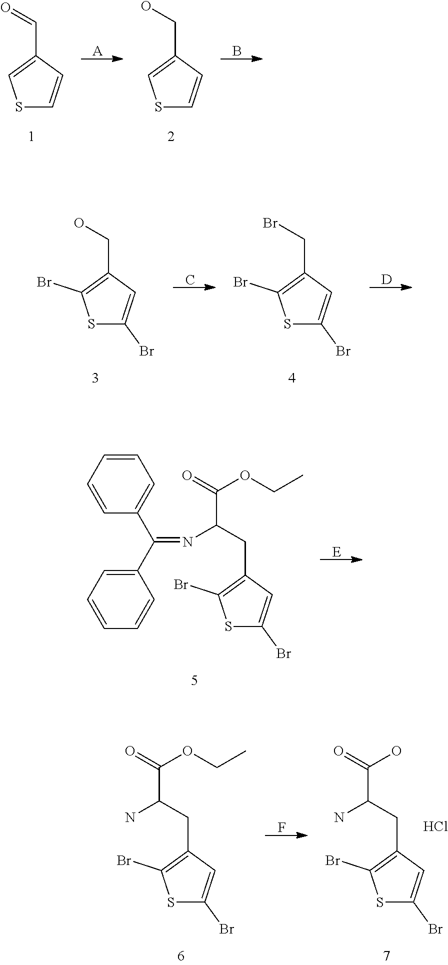

A. Sodium borohydride (2.0 g, 53 mmol, 0.5 eq.) was added to a solution of aldehyde 1 (12.0 g, 107 mmol, 1 eq.) in methanol (150 ml) at 0° C. The reaction mixture was stirred 4 hours at room temperature and then partitioned in 5% HCl (300 mL)/and ethyl acetate (150 mL). The aqueous layer was separated and extracted with ethyl acetate (2×150 ml). The combined organic phases were dried over Na2SO4, filtered and concentrated to dryness to give the product 2 (10.1 g, 83%)

B. NBS (30.7 g, 173 mmol, 1.95 eq.) was added gradually to compound 2 (10.1 g, 88 mmol, 1 eq.) in THF (300 mL) and the mixture was kept at RT overnight, filtered, and then THF was removed by rotary evaporation. The product was dissolved in diethyl ether, then rinsed with 1M sodium hydroxide solution and water. The organic layer was dried over Na2SO4 and the product was eluted over silica gel using hexane:ethyl acetate (80:20). The solvent was removed by rotary evaporation to yield the desired product 3 (18.5 g, 77% yield).

C. Compound 3 (17.3 g, 64 mmol, 1 eq.) in DCM (200 mL) was added to a 500 ml flask, and the flask was placed in an ice water bath. Phosphorus tribromide (6.6 ml, 70 mmol, 1.1 eq.) was added dropwise to the solution. The reaction was run at room temperature for 5 h, then quenched with a 10% sodium bicarbonate solution. The organic solution was washed with water, dried over Na₂SO₄ anhydride and concentrated under reduced pressure to give compound 4 (18.5 g, 87% yield).

D. To a flame-dried flask containing t-BuOK (6.8 g, 61 mmol, 1.10 eq.) and anhydrous DMF (500 ml) was added ethyl 2-((diphenylmethylene)amino)acetate 2 (15.5 g, 58 mmol, 1.05 eq.) at 0° C. under argon. After 10 min, bromide 4 (18.5 g, 55 mmol, 1.0 eq.) was added. The resulting reaction mixture was stirred at 0° C. for 10 min and was allowed to warm up to room temperature and stirred overnight, then slowly poured into water, extracted with DCM. The combined organic layers were dried over Na₂SO₄ anhydride, filtered and concentrated under reduced pressure to give compound 5 (21.3 g, 74%).

E. Compound 5 (21.3 g, 41 mmol) was treated with a mixture of EtOH/concentrated HCl (25:1 v/v) (400 ml). The resulting solution was stirred at room temperature for 2 h. Upon the evaporation of solvent, the residue was basified by 2N K₂CO₃ solution to pH >10 and extracted with DCM. The combined organic layers were dried over Na2SO4, filtered and concentrated under reduced pressure. The residue was purified by flash chromatography on silica gel (1:20 CH3OH/DCM) to produce the product 6 (11.0 g, 75%).

F. Product 6 (11.0 g, 31 mmol) was dissolved in 3N hydrochloric acid (250 ml). The mixture was stirred at 70° C. for 12 h. The solvent was evaporated under reduced pressure to give the title compound (11.25 g, 99%).

Analytical data: ₁H NMR (DMSO-d₆) δ_{H} (ppm): 3.08 (m, 2H, CH₂), 4.05 (t, 1H, CH), 7.24 (s, 1H, HetAr.H); 8.2-9.1 (br.s, 2H, NH₂); EδI: Calculated [M₊] (C₇H₇O₂NSBr₂) m/z: 328.86. Found [M+H₊]=329.8 and 327.8, [M−H₊], 327.80 and 325.8.0.

### Example 4: Synthesis of 5-(2-amino-2-carboxyethyl)-1-methylpyrimidin-1-ium (Code 676)

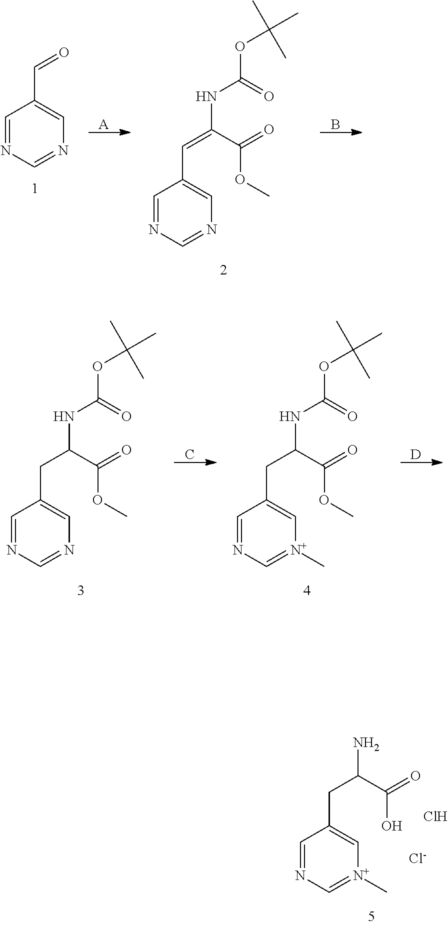

A. Compound 1 and methyl 2-{[(tert-butoxy)carbonyl]amino}-2-(dimethyl phosphonoacetate) (1.2 eq) were dissolved in DCM. Then reaction mixture was cooled and 2H,3H,4H,6H,7H,8H,9H,10H-pyrimido[1,2-a]azepine (1.4 eq) was slowly added. The reaction was stirred overnight at RT. After that it was washed with water (3 times) and brine, dried over NA₂SO₄ anhydride and evaporated to produce crude compound 2.

B. Compound 2 in MeOH was added to Pd/C, 5% (0.1 eq). The reaction mixture was stirred at RT overnight in H2 atmosphere, filtered and resulting solution was evaporated to produce crude compound 3. Crude compound 3 purified with column chromatography (Hex-EtOAc was used as an eluent mixture).

C. Compound 3 was dissolved in MeCN and MeI (7 eq) was added dropwise. The reaction was stirred at RT overnight. After that it was evaporated to produce crude compound 4 which was used in next step without further purification.

D. Compound 4 was dissolved in HCl/dioxane and the reaction was refluxed overnight. After that it was evaporated to afford crude compound 5 which was stirred with MeCN and filtered on, dried to afford target compound.

Analytical data: ₁H NMR (DMSO-d₆), δ_{H} (ppm): 3.40-3.60 (d, 2H, CH₂), 4.50-4.60 (br.s, 1H, CH), 8.80-8.95 (br.s, 3H, CH₃), 9.40 (s, 1H, PyrH), 9.65 (s, 1H, PyrH), 9.70 (s, 1H, PyrH). ₁₃C NMR (DMSO-d₆) δ_{H} (ppm): 30.12 (CH2), 45.5 (CH3), 52.3 (CH), 132.2 (PyrCq), 153.0 (PyrCH), 154.3 (PyrCH), 165.0 (PyrCH), 169.8 (COOH). EδI: Calculated [M₊] (C₈H₁₂O₂N₃) m/z: 182.0. Found [M−e₋]₊, 182.0.

### Example 5: Synthesis of methyl 2-amino-3-(2,6-dibromopyridin-4-yl)propanoate (Code 670)

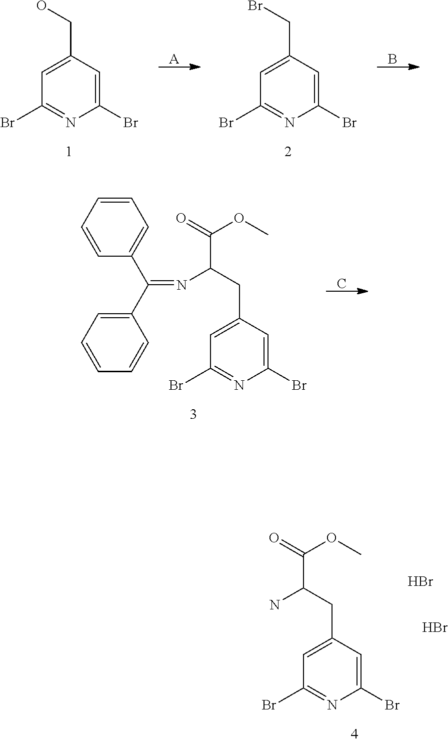

A. Compound 1 (16.0 g, 60 mmol, 1 eq.) in DCM (200 mL) was added to a 500 ml flask, and the flask was placed in an ice water bath. Phosphorus tribromide (6.8 ml, 72 mmol, 1.2 eq.) was added dropwise to the solution. The reaction was run at room temperature for 5 h, then quenched with a 10% sodium bicarbonate solution. The organic solution was washed with water, dried over Na₂SO₄ and concentrated under reduced pressure to give compound 2 (18.5 g, 94.0% yield).

B. To a flame-dried flask containing t-BuOK (6.9 g, 62 mmol, 1.10 eq.) and anhydrous DMF (500 ml) was added methyl 2-((diphenylmethylene)amino) acetate (14.9 g, 59 mmol, 1.05 eq.) at 0° C. under argon. After 10 min, bromide 2 (18.5 g, 55 mmol, 1.0 eq.) was added. The resulting reaction mixture was stirred at 0° C. for 10 min and was allowed to warm up to room temperature and stirred overnight. The reaction mixture was then slowly poured into water, extracted with DCM. The combined organic layers were dried over NA₂SO₄ anhydride, filtered and concentrated under reduced pressure to give compound 3 (24.5 g, 87.0%).

C. Compound 3 (24.5 g, 49 mmol) was treated with a mixture of EtOH/48% HBr (25:1 v/v) (600 ml). The resulting solution was stirred at room temperature for 2 h. After evaporation of solvent the residue was rinsed with water, washed with hexane and concentrated under reduced pressure to produce the target product 4 (19.7 g, 81.0%).

Analytical data: ₁H NMR (DMSO-d₆), δ_{H} (ppm): 3.00-3.10 (m, 1H, CH₂), 3.15-3.23 (m, 1H, CH₂), 3.75 (s, 3H, CH₃), 4.50-4.58 (t, 1H, CH), 7.70 (s, 2H, PyrH), 8.30-8.40 (br.s, 3H, NH₂.H₊); EδI: Calculated [M₊] (C₉H₁₀O₂N₂Br₂) m/z: 338.0 (Br 79 and 81). Found [M−e₋]₊, 339.0 and 341.0.

### Example 6: Synthesis of ethyl 2-amino-3-[(2,5-dibromothiophene-3-sulfonyl)amino]propanoate (Code 580)

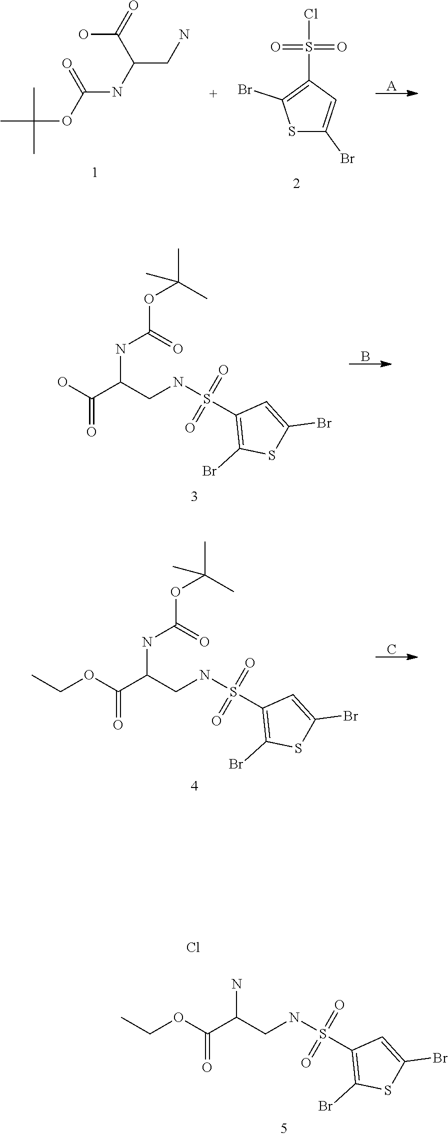

Step A N-Boc-3-amino-alanin 1 (50 g, 0.23 mol, 2.3 eq) was suspended in 2 l of dry acetone in the flame dried 4 l flask under argon. Then, TEA (70 ml, 0.5 mol, 5.1 eq) was added and reaction mixture stirred for 30 minutes at RT. Compound 2 was dissolved in dry acetone and dropped into the reaction mixture at −5−+5° C. Reaction mixture was stirred at RT overnight. Then reaction mixture was poured to the ice, acidified with water solution of citric acid and extracted with ethyl acetate (5×500 ml). Combined organic layers were washed with water (2×200 ml), dried over NA₂SO₄ anhydride and evaporated under reduced pressure. Obtained crude compound 3 was purified by column chromatography. Mass of isolated product 3 (90% purity) (9 g, yield 18%).

Step B. DCC (2.6 g 0.0124 mol, 1.1 eq) and DMAP (0.014 g 0.00114 mol, 0.01 eq), were treated with solution of sulfamide 3 (5.8 g, 0.0114 mol, 1 eq) in 500 ml of dry methylene chloride in the flame dried 1 l 3-neck flask. After that, dry ethanol (˜2 ml, 0.022 mol, ˜2 eq) was added to the reaction mixture. Reaction mixture was stirred at RT overnight. Then reaction mixture was evaporated under reduced pressure. Water was added to the residue and target compound was extracted with ethyl acetate. Combined organic layers were dried Na₂SO₄ and evaporated. Crude compound was purified by column chromatography (Yield 2.5 g of 90% purity of target compound, 40% yield).

Step C. HCl\dioxane (100 ml) was added to the sulfamide 4 (2.5 g, 0.0046 mol, 1 eq) dissolved in 20 ml of dry chloroform in the flame dried 250 ml flask. Reaction mixture stirred overnight. Solvent was evaporated. The target compound was purified by HPLC. Yield 0.7959 g (˜36%).

Analytical data: Solid ₁H NMR δH (ppm): 1.28-1.37 (t, 3H, CH₃), 3.35-3.45 (m, 1H, CH₂), 3.51-3.55 (m, 1H, CH₂), 4.04-407 (br.s, 1H, CH), 4.20-4.30 (m, 2H, CH₂), 7.30 (s, 1H, Thiop.H); EδI: Calculated [M]₊ (C₉H₁₂O₄N₂S₂Br₂) m/z=404 (Br 79/81) and 406 (Br=81). Found [M+H₊]=406.8 (Br=79/81) and 468.8 (Br81/81).

### Example 7: Synthesis of ethyl 3-(2,5-dichlorothiophen-3-yl)-2-acetamidopropanoate (Code 573)

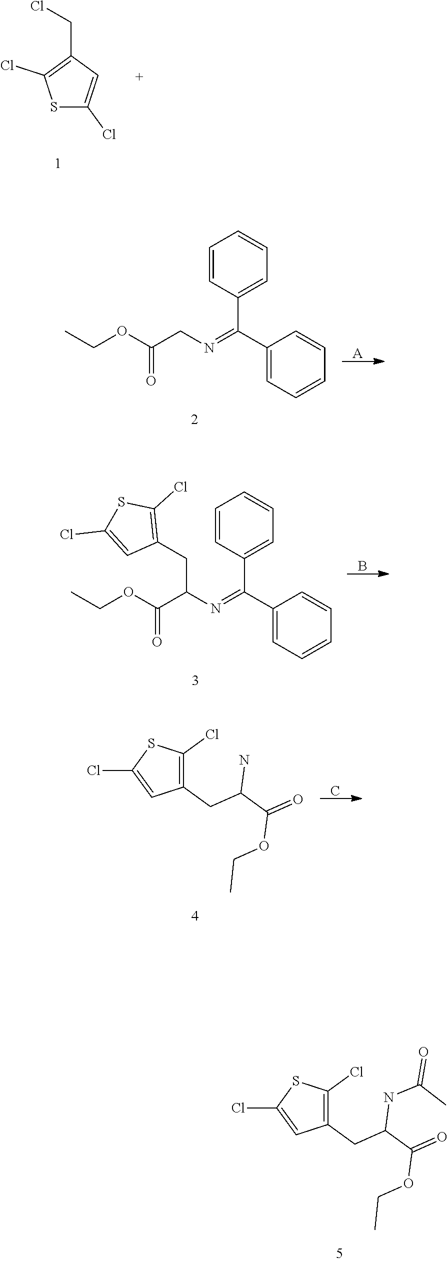

A. To a flame-dried flask containing t-BuOK (9.2 g, 82 mmol, 1.10 eq.) and anhydrous DMF (700 ml) was added ethyl 2-((diphenylmethylene)amino) acetate 2 (20.9 g, 78 mmol, 1.05 eq.) at 0° C. under argon. After 10 min, chloride 1 (15 g, 74 mmol, 1.0 eq.) was added. The resulting reaction mixture was stirred at 0° C. for 10 min and then the mixture was stirred overnight at RT. The reaction mixture was then slowly poured into water, extracted with DCM. The combined organic layers were dried over NA₂SO₄ anhydride, filtered and concentrated under reduced pressure to give compound 3 (21 g, 65.2%).

B. Compound 3 (21.0 g, 49 mmol) was treated with a mixture of EtOH/concentrated HCl (25:1 v/v) (500 ml). The resulting solution was stirred at room temperature for 2 h. Upon the evaporation of solvent, the residue was basified by 2N K2CO3 solution to pH >10 and extracted with DCM. The combined organic layers were dried over Na2SO4, filtered and concentrated under reduced pressure. The residue was purified by flash chromatography on silica gel (1:20 CH3OH/DCM) to give compound 4 (12.7 g, 86%).

C. To a stirred solution of compound 4 (12.7 g, 47 mmol, 1.0 eq.) in 300 mL of DCM was added 13.2 ml (95 mmol, 2 eq.) of TEA followed by 4.9 ml (52 mmol, 1.1 eq.) of acetic anhydride. The reaction mixture was stirred at room temperature for 12 h. The reaction mixture was then diluted with 150 ml of NaHCO3 and extracted with DCM. The combined organic layers were dried over Na2SO4, filtered and concentrated under reduced pressure. The residue was purified by flash chromatography on silica gel (1:4 EtOAc/Hexane) to give compound 5 (13.3 g, 91%).

Analytical data: ₁H NMR (400 MHz, CDCl₃) δ_{H} (ppm): 1.2-1.3 (t, 3H, CH₃), 1.95 (s, 3H, CH₃), 2.95-3.10 (dd, 2H, CH₂-thiophen), 4.10-4.25 (m, 2H, CH₂-ethyl), 4.75-4.80 (d, 1H, CH), 6.00 (s, 1H, H-thiophen), 6.60 (s, 1H, NH); EδI: Calculated [M₊] (C₁₁H₁₃O₃NSCl₂) m/z: 310.0 and 312.0. Found [M−e₋]₊, 310.0 and 312.0.

### Example 8: Synthesis of 2-ethoxyethyl 3-(dichloro-1,3-thiazol-4-yl)-2-acetamidopropanoate (Code 555)

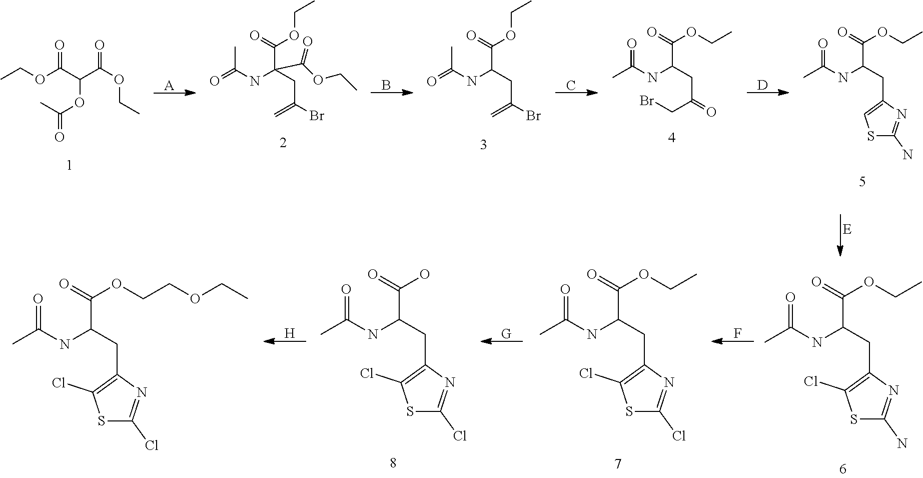

To a stirred mixture of NaH (44 g, 1.1 mol, 60% in silicone oil) in dry DMF (2.50 L) were added diethyl acetamidomalonate 1 (217 g, 1.0 mol) and 2,3-dibromopropene (240 g, 1.2 mol). The reaction mixture was stirred at room temperature overnight. After the reaction was complete (controlled by HNMR) the mixture was used in the next step without quenching.

B. To a soln of diethyl 2-acetamido-2-(2-bromoallyl)malonate 2 (336 g) in DMF (2.5 L) H2O (45 mL, 2.5 mol) and LiBr (102 g, 1.2 mol) were subsequently added and the mixture was heated to reflux under argon for 6 h. The mixture was subsequently evaporated to remove DMF and quenched with EtOAc (1.5 L) and H2O (1 L). The mixture was stirred for 10 min and extracted with EtOAc (3×0.5 L), the combined organic layers were washed with brine (3×0.5 L), dried over Na2SO4 and concentrated in vacuo. Obtained oily residue 3 (210 g, 93% purity) was used in the next step without purification.

C. To a cold (0° C.) stirred solution of 3 (210 g, 93% purity, 0.74 mol), under nitrogen, in a 2:1 mixture of acetonitrile/water (1.68 L) was added solid N-bromosuccinimide (193 g, 1.08 mole) in three portions over a period of 15 min. The resultant orange mixture was stirred at 0° C. for an additional period of 1 h and then allowed to warm to room temperature. After 4 h, the reaction mixture was treated with 10% aqueous sodium thiosulfate, diluted with ethyl acetate (1.5 L) and washed with water (4×300 mL) and brine (4×300 mL). Evaporation of the organic solvents results in the compound 4 (122 g, 0.43 mol) which was used in the next step without purification.

D. Compound 4 (122 g, 0.43 mol) and thiourea (33 g, 0.44 mol) were dissolved in EtOH (1.5 L) and stirred under reflux overnight. After the reaction was complete (confirmed by HNMR), the organic solvents were evaporated, and the crude residue was partitioned in EtOAc/H2O (1 L:1 L). The lower layer was carefully basified with 10% aq NaOH to pH=9 and extracted with CH2Cl2 (3×150 mL). Combined organic layers was washed with brine (2×100 mL), dried over Na2SO4 and evaporated to give product 5 (80 g, 0.28 mol)

E. A solution of 5 (80 g, 0.28 mol) and N-chlorosuccinimide (56 g, 0.42 mol) in acetonitrile (1.5 L) was stirred at RT for one hour. After the completion of the reaction, acetonitrile was evaporated under reduced pressure and the residue was partitioned in water (400 mL) and ethyl acetate (400 mL). The layers were separated, and the aqueous layer was extracted with ethyl acetate (3×150 mL). The combined organic layers were washed with brine, dried over sodium sulfate, filtered and concentrated to give product 6 (60 g, crude, 70% purity) which was used immediately in the next step without purification.

F. To a round bottomed flask was added crude compound 6 (60 g, crude, 70% purity), copper (II) chloride (38 g, 0.28 mol), and tert-butyl nitrite (35.6 mL, 0.3 mol) in CH₃CN (1 L). The mixture was heated to 40° C. for 2 hours, then the mixture was diluted with 1N HCl and extracted with EtOAc. The organic extract was washed with water (2×100 mL), brine (2×100 mL), dried over Na2SO4, filtered and concentrated in vacuo to give compound 7, ethyl 2-acetamido-3-(2,5-dichlorothiazol-4-yl) propanoate. The product was purified by CC (5.5 g, 0.018 mol)

G. To the solution of 7 (5.5 g, 0.018 mol) in CH3OH (30 mL) KOH (1.1 g, 0.0198 mol) in water (5 mL) was added in few portions and the resulting mixture was stirred overnight. After the reaction was complete (confirmed by HNMR), the organic solvent was evaporated, and the crude residue was partitioned in MTBE/H2O (30 mL:50 mL). The organic layer was discarded; the water layer was acidified with 10% aq. NaHSO4 to pH=3 and extracted with DCM (3*50 mL). Combined organic layer was dried over Na2SO4 and evaporated under reduced pressure to give compound 8 (3.2 g, 0.011 mol), which was used in the next step without purification.

H. 2-acetamido-3-(2,5-dichlorothiazol-4-yl)propanoic acid 8 (3.2 g, 0.011 mol) was dissolved in DCM (30 mL), followed by CDI (1.9 g, 0.012 mol) added in a few portions. After gas evolution stopped, 2-ethoxyethan-1-ol (5.3 mL, 0.055 mol) was added and the mixture was stirred overnight. After the reaction was complete, the mixture was diluted with DCM (50 mL), washed with 1 N HCl (3*15 mL), sat aq NaHCO3 (3*20 mL), brine (2*10 mL), dried over Na2SO4 and evaporated under reduced pressure. Crude product was purified with CC to give target compound as beige solid (1.5 g, 4.2 mmol)

Analytical data: ₁H NMR (CDCl₃), δ_{H} (ppm): 1.20 (t, 3H, CH₃), 2.01 (s, 3H, CH₃), 3.22 (d, 2H, CH₂), 3.50 (d, 2H, CH₂), 3.63 (t, 2H, CH₂), 4.28 (t, 2H, CH₂), 4.95 (d, 1H, CH), 6.40 (br.s, 1H, NH); EδI: Calculated [M] (C₁₂H₁₆, O₄, N₂SCl₂), m/z=354 (Cl=35.0), 356 (1Cl=35.0 and 2Cl=35.6). Found [M+H₊]=355.1.

### Example 9: Testing Post-Emergency Herbicidal Activity of Selected Non-Coding Heterocyclic Amino Acids (NCHAA)

The tests were performed on Romaine lettuce (Lactuca sativa L.) plants at the stage 2 to 3 leaves. The plants were grown on 300 well trays packed with soil, well dimensions 2×2×5 cm. The test dilution containing a test material listed in Tables 1, 2 & 3. Each compound was dissolved in 0.1% water solution of agronomically acceptable surfactant Silwet to final concentrations ranging from 0.0% (Control) to 0.5%. Two weeks after the treatment the herbicidal effects of the respective test material were visually evaluated by a comparison with the control plants. In addition, the aboveground part of the plant was neatly cut and weighed (wet weight). Each test was performed on 20 to 40 plants. Tested compounds are listed in Table 1.

TABLE 1
chemical and code names of the tested compounds

| Code | Chemical Name |
|---|---|
| 501 | 2-amino-3-(thiophen-2-yl)propanoic acid |
| 502 | 2-amino-3-(thiophen-3-yl)propanoic acid |
| 504 | 2-amino-3-[(thiophene-3-sulfonyl)amino]propanoic acid |
| 506 | 2-amino-3-[(4-fluorothiophene-3-sulfonyl)amino]propanoic acid |
| 507 | methyl 2-amino-3-[(thiophene-3-sulfonyl)amino]propanoate |
| 508 | pantyl 2-amino-3-[(thiophene-3-sulfonyl)amino]propanoate |
| 509 | 3-(5-cyano-3-thienyl)alanine |
| 510 | 2-amino-3-[4-(trifluoromethyl)thiophen-2-yl]propanoic acid |
| 511 | 3-amino-3-(4-bromothiophen-3-yl)propanoic acid |
| 512 | 2-amino-3-(2,4,5-trimethylthiophen-3-yl)propanoic acid |
| 513 | butyl 2-amino-3-(thiophen-3-yl)propanoate |
| 514 | 2-amino-3-[(2,5-dichlorothiophene-3-sulfonyl)amino]propanoic acid |
| 515 | 2-amino-3-[(2,5-dichloro-4-methylthiophene-3-sulfonyl)amino]propanoic acid |
| 516 | 2-amino-3-(2-chlorothiophen-3-yl)propanoic acid |
| 517 | 2-amino-3-(2,5-dichlorothiophen-3-yl)propanoic acid |
| 518 | 2-amino-3-(5-chlorothiophen-3-yl)propanoic acid |
| 519 | 2-amino-3-(2,5-dihydro-1,3-thiazol-4-yl)propanoic acid |
| 520 | 2-amino-3-(1,3-thiazol-5-yl)propanoic acid |
| 521 | 2-amino-3-{[2,5-dichloro-4-(trifluoromethyl)thiophene-3-sulfonyl]amino}propanoic acid |
| 522 | 2-amino-3-(2-amino-1,3-thiazol-4-yl)propanoic acid |
| 523 | methyl 2-amino-3-[(2,5-dichlorothiophene-3-sulfonyl)amino]propanoate |
| 524 | ethyl 2-amino-3-[(2,5-dichlorothiophene-3-sulfonyl)amino]propanoate |
| 525 | 2-propoxyethyl 2-amino-3-[(2,5-dichlorothiophene-3-sulfonyl)amino]propanoate |
| 526 | [2-(carboxyamino)-3-ethoxy-3-oxopropyl](2,5-dichlorothiophene-3-sulfonyl)carbamic acid |
| 527 | 2-amino-3-(2,5-dichloro-1,3-thiazol-4-yl)propanoic acid |
| 528 | 2-amino-3-(5-chloro-1,3-thiazol-4-yl)propanoic acid |
| 529 | 2-amino-3-(1,2,5-thiadiazol-3-yl)propanoic acid |
| 532 | 2-amino-3-(1,2,4-thiadiazol-5-yl)propanoic acid |
| 534 | 2-amino-3-(furan-3-yl)propanoic acid |
| 542 | 2-amino-3-(1,3-oxazol-4-yl)propanoic acid |
| 545 | 2-formamido-3-(thiophen-3-yl)propanoic acid |
| 546 | methyl 2-amino-3-(thiophen-3-yl)propanoate |
| 547 | ethyl 2-amino-3-(thiophen-3-yl)propanoate |
| 548 | [2-amino-3-(thiophen-3-yl)propanamido]acetic acid |
| 549 | 2-amino-N-[2-(2-hydroxyethoxy)ethyl]-3-(thiophen-3-yl)propanamide |
| 550 | ethyl 2-butanamido-3-(dichloro-1,3-thiazol-4-yl)propanoate |
| 551 | 2-butoxyethyl 2-acetamido-3-(2,5-dichloro-1,3-thiazol-4-yl)propanoate |
| 552 | 2-amino-3-(2,5-dibromothiophen-3-yl)propanoic acid |
| 553 | 2-amino-3-(5-nitrothiophen-3-yl)propanoic acid |
| 554 | butyl 3-(dichloro-1,3-thiazol-4-yl)-2-acetamidopropanoate |
| 555 | 2-ethoxyethyl 3-(dichloro-1,3-thiazol-4-yl)-2-acetamidopropanoate |
| 556 | 2-amino-3-[5-(trifluoromethyl)thiophen-3-yl]propanoic acid |
| 557 | (2S)-2-amino-3-(3,5-dioxo-1,2,4-oxadiazolidin-2-yl)propanoic acid |
| 558 | 2-amino-3-[(2,5-dibromothiophene-3-sulfonyl)amino]propanoic acid |
| 559 | 2-amino-3-(5-chloro-2-methylthiophen-3-yl)propanoic acid |
| 560 | 2-amino-3-(2-bromothiophen-3-yl)propanoic acid |
| 561 | 2-amino-3-(1,1-dioxo-1l6-thiolan-3-yl)propanoic acid |
| 562 | 2-amino-3-(thiolan-3-yl)propanoic acid |
| 563 | [2-amino-2-(thiophen-3-yl)ethyl]phosphonic acid |
| 564 | 2-amino-3-(2,5-dimethyl-1,1-dioxo-1H-1l6-thiophen-3-yl)propanoic acid |
| 565 | 3-(2,5-dioxo-2,5-dihydrothiophen-3-yl)-2-(phosphonoamino)propanoic acid |
| 566 | 2-amino-3-[4-(trifluoromethyl)thiophen-3-yl]propanoic acid |
| 567 | ethyl 2-acetamido-3-(2,5-dichloro-1,3-thiazol-4-yl)propanoate |
| 568 | methyl 2-amino-3-(2,5-dichlorothiophen-3-yl)propanoate |
| 569 | ethyl 2-amino-3-(2,5-dichlorothiophen-3-yl)propanoate |
| 570 | 2-(2-methoxyethoxy)ethyl 2-amino-3-(thiophen-3-yl)propanoate |
| 571 | ethyl 2-acetamido-3-(thiophen-3-yl)propanoate |
| 573 | ethyl 3-(2,5-dichlorothiophen-3-yl)-2-acetamidopropanoate |
| 574 | ethyl 3-(2,5-dichlorofuran-3-yl)-2-acetamidopropanoate |
| 575 | propyl 2-amino-3-(2,5-dichlorofuran-3-yl)propanoate |
| 576 | 4-(2-acetamido-3-ethoxy-3-oxopropyl)-3-methyl-1,3-thiazol-3-ium chloride |
| 577 | 2-(2-ethoxyethoxy)ethyl 3-(2,5-dichlorothiophen-3-yl)-2-acetamidopropanoate |
| 578 | panty! 2-acetamido-3-(1,3,4-oxadiazol-2-yl)propanoate |
| 580 | ethyl 2-amino-3-[(2,5-dibromothiophene-3-sulfonyl)amino]propanoate |
| 595 | butyl 3-(5-chloro-1,2,4-thiadiazol-3-yl)-2-acetamidopropanoate |
| 599 | 2-amino-3-(1H-indazol-3-yl)propanoic acid hydrochloride[HCl] |
| 601 | 2-amino-3-(pyridazin-4-yl)propanoic acid |
| 602 | 2-amino-3-(pyridin-4-yl)propanoic acid |
| 603 | 2-amino-3-(pyridin-3-yl)propanoic acid dihydrochloride |
| 605 | 3-(5-Hydroxy-3-pyridinyl)alanine |
| 606 | 2-amino-3-(5-methylpyridin-3-yl)propanoic acid |
| 607 | 2-amino-3-(4-aminopyridin-2-yl)propanoic acid |
| 608 | 2-amino-3-(2-aminopyridin-4-yl)propanoic acid |
| 609 | 2-amino-3-(6-aminopyridin-2-yl)propanoic acid |
| 610 | 2-amino-3-(5-aminopyridin-3-yl)propanoic acid |
| 612 | 2-amino-3-(5-sulfanylpyridin-3-yl)propanoic acid |
| 613 | 2-amino-3-(2-sulfanylpyridin-4-yl)propanoic acid |
| 614 | 2-amino-3-(pyrimidin-5-yl)propanoic acid |
| 616 | 2-amino-3-(4-amino-6-chloropyrimidin-2-yl)propanoic acid |
| 618 | 2-amino-3-(pyrimidin-2-yl)propanoic acid dihydrochloride |
| 619 | 2-amino-3-(4-hydroxyl)yrimidin-2-yl)propanoic acid |
| 620 | 2-amino-3-(2,4-dioxo-1,2,3,4-tetrahydropyrimidin-5-yl)propanoic acid |
| 621 | 2-amino-3-(pyrazin-2-yl)propanoic acid |
| 622 | 2-amino-3-(5-chloropyridin-3-yl)propanoic acid |
| 624 | 2-amino-3-(2,5-dichloropyridin-3-yl)propanoic acid |
| 625 | 2-amino-3-(pyrimidin-4-yl)propanoic acid |
| 626 | 2-amino-3-(5-fluoropyridin-3-yl)propanoic acid |
| 627 | 2-amino-3-(6-chloropyridin-3-yl)propanoic acid |
| 628 | 2-amino-3-(2,6-dichloropyridin-4-yl)propanoic acid |
| 629 | 2-amino-3-(3,5-dichloropyridin-4-yl)propanoic acid |
| 630 | 2-amino-3-(3,5-difluoropyridin-4-yl)propanoic acid |
| 631 | 2-amino-3-[2-(dimethylamino)pyridin-4-yl]propanoic acid |
| 632 | 2-amino-3-(2-bromopyridin-4-yl)propanoic acid |
| 633 | 2-amino-3-(5,6-dibromopyridin-3-yl)propanoic acid |
| 634 | 2-amino-3-(2,6-dibromopyridin-4-yl)propanoic acid |
| 635 | 2-amino-3-(2,3-dibromopyridin-4-yl)propanoic acid |
| 637 | 2-amino-3[5-(trifluoromethyl)pyridin-3-yl]propanoic acid |
| 641 | 2-amino-3-(5,6-dichloropyridin-3-yl)propanoic acid |
| 642 | 2-amino-3-(2-oxo-1,2-dihydropyridin-4-yl)propanoic acid |
| 643 | 2-amino-3-[4-(dimethylamino)pyridin-3-yl]propanoic acid |
| 644 | 2-amino-3-(2-oxo-1,2-dihydropyridin-3-yl)propanoic acid |
| 645 | 2-amino-3-(6-oxo-1,6-dihydropyrimidin-4-yl)propanoic acid |
| 646 | 2-amino-3-(6-oxo-1,6-dihydropyrimidin-5-yl)propanoic acid |
| 650 | 2-amino-3-(6-oxo-1,6-dihydropyridin-3-yl)propanoic acid |
| 652 | 2-amino-3-(2,6-dioxo-1,2,3,6-tetrahydropyrimidin-4-yl)propanoic acid |
| 653 | 2-amino-3-(6-oxo-2-sulfanyl-1,6-dihydropyrimidin-4-yl)propanoic acid |
| 654 | propyl 2-amino-3-(2-bromopyridin-4-yl)propanoate |
| 655 | 2-amino-3-(2-bromo-3,5-difluoropyridin-4-yl)propanoic acid |
| 656 | propyl 2-amino-3-(2-bromo-3,5-difluoropyridin-4-yl)propanoate |
| 657 | 2-amino-3-[(2-bromopyridine-4-sulfonyl)amino]propanoic acid |
| 658 | propyl 2-amino-3-[(2-bromopyridine-4-sulfonyl)amino]propanoate |
| 661 | ethyl 2-amino-3-(5,6-dibromopyridin-3-yl)propanoate |
| 662 | ethyl 2-amino-3-(2,3-dibromopyridin-4-yl)propanoate acid |
| 663 | 2-amino-342-chloro-5-(trifluoromethyl)pyridin-3-yl]propanoic acid |
| 664 | 2-amino-3-(2-amino-6-oxo-1,6-dihydropyrimidin-5-yl)propanoic acid |
| 665 | 2-amino-3-(6-hydroxy-5-nitropyridin-3-yl)propanoic acid |
| 666 | methyl 2-amino-3-(2-bromopyridin-4-yl)propanoate |
| 667 | 3-(2-amino-2-carboxyethyl)-1-methylpyridin-1-ium |
| 668 | 3-(2-amino-2-carboxyethyl)-1-hydroxyl)yridin-1-ium |
| 669 | 2-amino-3-(4-amino-6-hydroxy-2-methylpyrimidin-5-yl)propanoic acid |
| 670 | methyl 2-amino-3-(2,6-dibromopyridin-4-yl)propanoate |
| 671 | ethyl 3-(6-bromopyridin-3-yl)-2-acetamidopropanoate |
| 672 | methyl 3-(6-bromopyridin-3-yl)-2-acetamidopropanoate |
| 673 | ethyl 2-acetamido-3-(2-methoxyl)yrimidin-4-yl)propanoate |
| 674 | 2-(2-methoxyethoxy)ethyl 2-amino-3-(6-bromopyridin-3-yl)propanoate |
| 675 | 2-(2-ethoxyethoxy)ethyl 2-amino-3-(6-bromopyridin-3-yl)propanoate |
| 676 | 5-(2-amino-2-carboxyethyl)-1-methylpyrimidin-1-ium |
| 679 | 2-amino-3[2-(trifluoromethyl)pyridin-4-yl]propanoic acid hydrochloride[HCl] |
| 682 | ethyl 3-[(2-bromopyridin-4-yl)oxy]-2-acetamidopropanoate |
| 683 | ethyl 2-acetamido-3-(pyridin-4-yloxy)propanoate |
| 702 | 2-amino-4-(1H-pyrrol-3-yl)butanoic acid |
| 703 | 2-amino-3-(1H-pyrrol-2-yl)propanoic acid |
| 704 | 2-amino-3-(1H-pyrrol-3-yl)propanoic acid |
| 709 | 2-amino-3-(4H-1,2,4-triazol-3-yl)propanoic acid |
| 710 | (2S)-2-hydroxy-3-(1H-imidazol-4-yl)propanoic acid |
| 714 | ethyl 2-{[(benzyloxy)carbonyl]amino}-3-(1H-1,2,4-triazol-3-yl)propanoate |
| 717 | 2-amino-3-(4-methyl-4H-1,2,4-triazol-3-yl)propanoic acid |
| 770 | 2-amino-3-(1-methyl-1H-1,2,4-triazol-5-yl)propanoic acid dihydrochloride |
| 771 | 2-amino-3-(1-methyl-1H-1,2,3-triazol-4-yl)propanoic acid hydrochloride |
| 772 | 2-amino-3-(2-methyl-2H-1,2,3,4-tetrazol-5-yl)propanoic acid hydrochloride |
| 773 | 2-amino-3-(dimethyl-4H-1,2,4-triazol-3-yl)propanoic acid hydrochloride |
| 774 | (2S)-2-amino-3[5-(trifluoromethyl)-1H-pyrazol-4-yl]propanoic acid |
| 775 | 2-(2-butoxyethoxy)ethyl 2-amino-3-(2H-1,2,3-triazol-4-yl)propanoate |

The degree of growth inhibition and damage was evaluated according to the parameters in Table 2. Plant development at 7, 14, and 21 DAA (days after application) were evaluated on a scale of 0 to 100, in which score 0 represents dead plants and score 100 represents healthy plants according to plant vigor, height, and leaf color. Each test included 20 to 40 plants.

TABLE 2
Activity Index

| Activity | Plant's development vs Control |
|---|---|
| Index | (% of Control) |
| Maximal | Less than 15 |
| Very Strong | From 15 to 25 |
| Strong | From 25 to 50 |
| Moderate | From 50 to 75 |
| Weak | More than 75 |

### Results:

The observed activity is demonstrated in Table 3.

TABLE 3
post-emergency herbicidal activity of tested compounds

| Code | Activity |
|---|---|
| 501 | Strong |
| 502 | Very Strong |
| 504 | Weak |
| 506 | Weak |
| 507 | Weak |
| 508 | Weak |
| 509 | Maximal |
| 510 | Strong |
| 511 | Weak |
| 512 | Weak |
| 513 | Very Strong |
| 514 | Weak |
| 515 | Weak |
| 516 | Maximal |
| 517 | Maximal |
| 518 | Strong |
| 519 | Moderate |
| 520 | Weak |
| 521 | Weak |
| 522 | Weak |
| 523 | Weak |
| 524 | Weak |
| 525 | Weak |
| 526 | Weak |
| 527 | Maximal |
| 528 | Weak |
| 529 | Moderate |
| 532 | Moderate |
| 534 | Strong |
| 542 | Weak |
| 545 | Weak |
| 546 | Very Strong |
| 547 | Very Strong |
| 548 | Strong |
| 549 | Weak |
| 550 | Maximal |
| 551 | Maximal |
| 552 | Maximal |
| 553 | Weak |
| 554 | Maximal |
| 555 | Maximal |
| 556 | Maximal |
| 557 | Weak |
| 558 | Very Strong |
| 559 | Moderate |
| 560 | Strong |
| 561 | Weak |
| 562 | Weak |
| 563 | Weak |
| 564 | Weak |
| 565 | Weak |
| 566 | Maximal |
| 567 | Maximal |
| 568 | Maximal |
| 569 | Maximal |
| 570 | Very Strong |
| 571 | Very Strong |
| 573 | Maximal |
| 574 | Moderate |
| 575 | Moderate |
| 576 | Maximal |
| 577 | Strong |
| 578 | Strong |
| 579 | Very Strong |
| 580 | Very Strong |
| 581 | Moderate |
| 594 | Strong |
| 595 | Strong |
| 601 | Weak |
| 602 | Moderate |
| 603 | Strong |
| 605 | Weak |
| 606 | Weak |
| 607 | Weak |
| 608 | Moderate |
| 609 | Weak |
| 610 | Weak |
| 612 | Weak |
| 613 | Weak |
| 614 | Weak |
| 616 | Weak |
| 618 | Weak |
| 619 | Weak |
| 620 | Weak |
| 621 | Weak |
| 622 | Moderate |
| 624 | Moderate |
| 625 | Weak |
| 626 | Strong |
| 627 | Moderate |
| 628 | Very Strong |
| 629 | Moderate |
| 630 | Strong |
| 631 | Weak |
| 632 | Maximal |
| 633 | Strong |
| 634 | Very Strong |
| 635 | Weak |
| 637 | Weak |
| 641 | Very Strong |
| 642 | Weak |
| 643 | Weak |
| 644 | Weak |
| 645 | Weak |
| 646 | Weak |
| 650 | Weak |
| 652 | Weak |
| 653 | Weak |
| 654 | Strong |
| 655 | Maximal |
| 656 | Very Strong |
| 657 | Weak |
| 658 | Weak |
| 661 | Moderate |
| 662 | Moderate |
| 663 | Moderate |
| 664 | Weak |
| 665 | Weak |
| 666 | Maximal |
| 667 | Weak |
| 668 | Weak |
| 669 | Weak |
| 670 | Very Strong |
| 671 | Weak |
| 672 | Moderate |
| 673 | Weak |
| 674 | Moderate |
| 675 | Moderate |
| 676 | Very Strong |
| 679 | Strong |
| 682 | on-going |
| 683 | on-going |
| 702 | Weak |
| 703 | Weak |
| 704 | Weak |
| 709 | Maximal |
| 710 | Weak |
| 712 | Maximal |
| 714 | Weak |
| 715 | Maximal |
| 716 | Moderate |
| 717 | Weak |
| 770 | Weak |
| 771 | Weak |
| 772 | Moderate |
| 773 | Weak |
| 774 | Very Strong |
| 775 | Maximal |

### Example 10: Testing Herbicidal Activity of Selected Non-Coding Heterocyclic Amino Acids (NCHAA) on the Development of Weeds and Crops

The experiment included 225 pots. Weed seeds were sawn in 0.3-liter pots, containing medium-heavy clay-loam soil from Newe Ya'ar Research Center organic fields (55% clay, 23% silt, 20% sand, 2% organic matter, pH 7.1). The experiment was conducted in a completely randomized design. Each treatment was replicated 5 times (5 pots). The herbicides were applied at 200 L/ha with a motorized sprayer equipped with a Tee Jet 8001E nozzle (Spraying Systems Co., North Ave., Wheaton, Ill. 60188, USA) and operated at a pressure of 300 kPa. The pots were transferred to a glasshouse 24 hours after herbicide application and were sprinkler irrigated to field capacity of the soil. The plants were grown in the glasshouse during the entire experiment. Plant development at 6, 12, 19, 26 and 32 days after application (DAA) were evaluated on a scale of 0 to 100%, in which 0 represents dead plants and 100% represents healthy plants according to plant vigor, height, and leaf color.

### Tested Weeds & Crops:

Phalaris minor (Cannarygrass); Setaria verticillata (Bristlegrass); Solanum nigrum (Black nightshade) Sinapis arvensis (Mustard); Silybum marianum (Thistle); Chenopodium album (Lambsquarter); Amaranthus (Pigweed); Fodder corn; Avena sativa (Cultured oats.

Plants development at 32 days after application (DAA) is shown in Table 4. Plant development was evaluated on a scale of 0 to 100%, in which 0 represents dead plants and 100% represents healthy plants according to plant vigor, height, and leaf color.

TABLE 4
Effect of NCHAA on the development of selected weeds & crops

| | Plants' Development (% of | |
|---|---|---|
| | Control group) following | |
| | treatment with NCHAAs | |
| Plant | Compound 632 | Compound 517 |

WEEDS

| | | |
|---|---|---|
| Solanum nigrum (Black nightshade) | 25% | 15% |
| Sinapis arvensis (Mustard) | 80% | 0% |
| Marianum (Thistle) | 15% | 35% |
| Amaranthus blitoides (Pigweed) | 100% | 10% |
| Chenopodium album (Lambsquarter) | 20% | 10% |
| Phalaris minor (Cannarygrass) | 10% | 60% |
| Setaria verticillata (Bristlegrass) | 20% | 100% |

CROPS

| | | |
|---|---|---|
| Avena sativa (Cultured oats) | 100% | 100% |
| Fodder Corn | 100% | 90% |

### Example 11: Testing Herbicidal Activity of Selected Non-Coding Heterocyclic Amino Acids (NCHAA) on Seeds Germination & Early Plants Development

Seeds of Lettuce R. (Super-Jericho, non-sterilized) were obtained from Ben Shahar Moshe Ltd., 99% purity. NCHAA were selected from compounds listed in the Table 1.

Filter Paper Bioassays with Lettuce Seeds:

The protocol is following Bertin et al. 2009 and Movellan et al. 2014 with specified modifications as described below.

Seeds were placed on Whatman no. 1 filter paper (Whatman, Middlesex, U.K.) in Petri dishes (10 seeds per plate) supplemented with 2.0 ml of water (Control) or with a solution comprising 1 mM, 0.1 mM, 0.01 mM and 0.001 mM of test material. Petri dishes with seeds were placed in a tray at an angle of 45°. The trays were kept in dark for 48 hours and then transferred to the growth chamber (22° C., 0.045 mmol×m−2×s−1 photosynthetic photon flux density; 6/18 dark/light cycle) for additional 4 days. Each experiment was performed at least in two repeats. The development of radicle and shoots of experimental and control plants were visually assessed 6 days after the beginning of the test to determine Minimal Effective Concentration (MEC) of test material. In the context of the invention, MEC is defined as the lowest concentration level of a test material that caused deviation (absence of germination or malformation of radicle and shoots) from the plant's development in the Control group. MEC is expressed in an arbitrary unit as activity score, as presented in Table 5:

TABLE 5
MEC as activity score

| MEC | Activity score |
|---|---|
| More than 1 mM | Weak |
| 1 mM | Moderate |
| 0.1 mM | Strong |
| 0.01 mM and less | Maximal |

TABLE 6
herbicidal activity of selected non-coding heterocyclic amino
acids (NCHAA) on seeds germination & early plants development

| Code | Activity |
|---|---|
| 501 | Maximal |
| 502 | Maximal |
| 504 | Weak |
| 506 | Weak |
| 507 | Weak |
| 508 | Weak |
| 509 | Strong |
| 511 | Weak |
| 512 | Weak |
| 513 | Strong |
| 514 | Weak |
| 515 | Weak |
| 516 | Maximal |
| 517 | Maximal |
| 519 | Maximal |
| 520 | Strong |
| 521 | Weak |
| 522 | Weak |
| 523 | Weak |
| 524 | Weak |
| 525 | Weak |
| 526 | Weak |
| 527 | Moderate |
| 528 | Moderate |
| 529 | Strong |
| 532 | Strong |
| 534 | Moderate |
| 542 | Weak |
| 545 | Weak |
| 546 | Maximal |
| 547 | Maximal |
| 549 | Weak |
| 552 | Moderate |
| 553 | Weak |
| 555 | Strong |
| 556 | Strong |
| 557 | Weak |
| 558 | Maximal |
| 561 | Weak |
| 562 | Weak |
| 563 | Weak |
| 564 | Weak |
| 565 | Strong |
| 566 | Strong |
| 567 | Weak |
| 569 | Strong |
| 570 | Maximal |
| 571 | Strong |
| 573 | Strong |
| 574 | Strong |
| 575 | Strong |
| 576 | Moderate |
| 577 | Moderate |
| 579 | Strong |
| 580 | Strong |
| 581 | Moderate |
| 595 | Strong |
| 599 | Maximal |
| 601 | Weak |
| 602 | Strong |
| 603 | Strong |
| 605 | Weak |
| 606 | Weak |
| 607 | Weak |
| 608 | Moderate |
| 609 | Weak |
| 610 | Weak |
| 612 | Weak |
| 613 | Weak |
| 614 | Weak |
| 616 | Weak |
| 618 | Weak |
| 619 | Weak |
| 620 | Weak |
| 621 | Weak |
| 622 | Strong |
| 624 | Moderate |
| 625 | Moderate |
| 626 | Strong |
| 627 | Strong |
| 628 | Moderate |
| 629 | Weak |
| 630 | Moderate |
| 631 | Weak |
| 632 | Strong |
| 633 | Strong |
| 634 | Strong |
| 635 | Weak |
| 637 | Strong |
| 639 | Strong |
| 640 | Weak |
| 641 | Strong |
| 642 | Weak |
| 643 | Weak |
| 644 | Weak |
| 645 | Weak |
| 646 | Weak |
| 650 | Moderate |
| 652 | Weak |
| 653 | Weak |
| 654 | Strong |
| 655 | Strong |
| 656 | Strong |
| 657 | Weak |
| 658 | Weak |
| 661 | Maximal |
| 662 | Strong |
| 663 | Strong |
| 664 | Moderate |
| 665 | Moderate |
| 666 | Strong |
| 667 | Weak |
| 668 | Weak |
| 669 | Weak |
| 670 | Strong |
| 671 | Strong |
| 672 | Strong |
| 673 | Weak |
| 674 | Strong |
| 675 | Strong |
| 676 | Moderate |
| 679 | Strong |
| 702 | Weak |
| 703 | Weak |
| 704 | Weak |
| 709 | Moderate |
| 710 | Weak |
| 712 | Moderate |
| 714 | Moderate |
| 715 | Moderate |
| 716 | Moderate |

### Discussion:

Design of non-coding heterocyclic amino acids (NCHAA) was intended to mimic natural aromatic amino acids, such as phenylalanine, tyrosine, histidine and tryptophan. These compounds were tested in a range of experimental setups, such as on seeds placed on the filter paper as well as in experiments with pre- or post-emergency treatment of weeds and crops. The experimental data suggest that, despite diversity of chemical structures, NCHAA as a group possesses a significant herbicidal activity. Surprisingly, selected NCHAAs demonstrated herbicidal activity significantly surpassing natural (such as m-Tyrosine) and synthetic non-coded amino acids with “non-heterocyclic, all carbon” aromatic rings, such as m-tyrosine compounds described by U.S. Pat. No. 8,461,085 (Leslie A. Weston et. al, Cornell Business & Technology Park). NCHAA appeared as a new family of compounds useful for crop protection, in particularly in crop protection used to destroy unwanted vegetations.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will prevail. In addition, the materials, methods, and examples are illustrative only and are not intended to be necessarily limiting.

As used herein the terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

For example, the term “a compound” or “at least one compound” may include a plurality of compounds, including mixtures thereof.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

As will be appreciated by those of skill in the art, the compounds of the various formulas disclosed herein may contain chiral centers, e.g., asymmetric carbon atoms. Thus, the present disclosure is concerned with the synthesis of both: (i) racemic mixtures of the active compounds, and (ii) enantiomeric forms of the active compounds. The resolution of racemates into enantiomeric forms and racemization of optically active enantiomeric form can be done in accordance with known procedures in the art. Geometric isomers of double bonds and the like may also be present in the compounds disclosed herein, and all such stable isomers are included within the present disclosure unless otherwise specified. Also included in the compounds of the disclosure are tautomers (e.g., tautomers of triazole and/or imidazole) and rotamers. All chains defined by the formulas herein which include three or more carbons may be saturated or unsaturated unless otherwise indicated.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

An “optionally substituted” group refers to a functional group in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Where multiple substituent moieties are disclosed or claimed, the substituted compound can be independently substituted by one or more of the disclosed or claimed substituent moieties, singly or pluraly.

By independently substituted, it is meant that the (two or more) substituents can be the same or different. In choosing the compounds of the present invention, one of ordinary skill in the art will recognize that the various substituents are to be chosen in conformity with well-known principles of chemical structure connectivity.

As used herein, “H” refers to a hydrogen atom. “C” refers to a carbon atom. “N” refers to a nitrogen atom. “O” refers to an oxygen atom. “Halo” refers to F, Cl, Br or I. The term “hydroxy,” as used herein, refers to an —OH moiety. “Br” refers to a bromine atom. “Cl” refers to a chlorine atom. “I” refers to an iodine atom. “F” refers to a fluorine atom. An “acyl group” is intended to mean a group —C(O)—R, where R is a suitable substituent, for example, an acetyl group, a propionyl group, a butyroyl group, a benzoyl group, or an alkylbenzoyl group. “Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). In some embodiments the alkyl can be a lower alkyl. “Lower alkyl” refers to straight or branched chain alkyl having from 1 to 3, or from 1 to 5, or from 1 to 8 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. As used herein, the identification of a carbon number range, e.g., C1-C12 alkyl, is intended to include each of the component carbon number moieties within such range, so that each intervening carbon number and any other stated or intervening carbon number value in that stated range is encompassed, such that sub-ranges of carbon number within specified carbon number ranges may independently be specified. For example, C1-C12 alkyl is intended to include methyl, ethyl, propyl, butyl, pentyl, hexyl, heptyl, octyl, nonyl, decyl, undecyl and dodecyl, including straight chain as well as branched groups, as noted above, and the carbon number range C1-C12 alkyl may also be more restrictively specified as sub-ranges such as C1-C4 alkyl, C2-C8 alkyl, C2-C4 alkyl, C3-C5 alkyl, or any other sub-range within the broader carbon number range. In addition, ranges of carbon numbers specifically excluding a carbon number or numbers are contemplated, as are sub-ranges excluding either or both of carbon number limits of specified ranges. As generally understood by those of ordinary skill in the art, “saturation” refers to the state in which all available valence bonds of an atom (e.g., carbon) are attached to other atoms. Similarly, “unsaturation” refers to the state in which not all the available valence bonds are attached to other atoms; in such compounds the extra bonds usually take the form of double or triple bonds (usually with carbon). For example, a carbon chain is “saturated” when there are no double or triple bonds present along the chain or directly connected to the chain (e.g., a carbonyl), and is “unsaturated” when at least one double or triple bond is present along the chain or directly connected to the chain (e.g., a carbonyl). Further, the presence or absence of a substituent depending upon chain saturation will be understood by those of ordinary skill in the art to depend upon the valence requirement of the atom or atoms to which the substituent binds (e.g., carbon). “Alkenyl,” as used herein, refers to a straight or branched chain hydrocarbon containing from 1 or 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Alkynyl,” as used herein, refers to a straight or branched chain hydrocarbon group containing from 1 or 2 to 10 or 20 or more carbon atoms, and containing at least one carbon-carbon triple bond. Representative examples of alkynyl include, but are not limited, to acetylenyl, 1-propynyl, 2-propynyl, 3-butynyl, 2-pentynyl, 1-butynyl and the like. The term “cycloalkyl,” as used herein, refers to a saturated cyclic hydrocarbon group containing from 3 to 8 carbons or more.

As understood in the art, the term “optionally substituted” indicates that the specified group is either unsubstituted or substituted by one or more suitable substituents. A “substituent” that is “substituted” is an atom or group which takes the place of a hydrogen atom on the parent chain or cycle of an organic molecule. “Heterocycle,” as used herein, refers to a monocyclic, bicyclic or tricyclic ring system. Monocyclic heterocycle ring systems are exemplified by any 5 to 9-membered ring containing 1, 2, 3, or 4 heteroatoms independently selected from the group consisting of: O, N, and S. “Aryl” as used herein refers to a ring system having one or more aromatic rings. Representative examples of aryl include azulenyl, indanyl, indenyl, naphthyl, phenyl, tetrahydronaphthyl, and the like. “Heteroaryl” means a cyclic, aromatic hydrocarbon in which one or more carbon atoms have been replaced with heteroatoms (e.g., N, O or S). If the heteroaryl group contains more than one heteroatom, the heteroatoms may be the same or different. “Alkoxy,” as used herein, refers to an alkyl group, as defined herein, appended to the parent molecular moiety through an oxy group, as defined herein. An “amine” or “amino” is intended to mean the group —NH₂. “Primary amines” have one of three hydrogen atoms replaced by an alkyl or aromatic group. “Secondary amines” have two organic substituents bound to the nitrogen together with one hydrogen. “Tertiary amines” have three organic substituents bound to the nitrogen. An “amide” as used herein, refers to a functional group having a carbonyl group (C═O) linked to a nitrogen atom (N), or an organic compound that contains this group, generally depicted as:

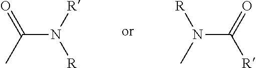

wherein, R and R′ can independently be any covalently linked atom or atoms. The term “oxo,” as used herein, refers to a ═O moiety. The term “oxy,” as used herein, refers to a —O— moiety. “Nitro” refers to the organic compound functional group —NO2. “Carbonyl” is a functional group having a carbon atom double-bonded to an oxygen atom (—C═O). “Carboxy” as used herein refers to a —COOH functional group, also written as —CO2H or —(C═O)—OH.

It will be understood that the compounds, compositions and methods provided herein may be further specified in some embodiments by provisos or limitations excluding specific substituents, groups, moieties, structures, ingredients, steps, or conditions, as applicable, in relation to various broader specifications and exemplifications set forth herein.

“Agriculturally acceptable carriers” of the invention include, without limitation, adjuvants, mixers, enhancers, etc. beneficial for application of the chemical formula. Suitable carriers should not be phytotoxic to valuable crops, particularly at the concentrations employed in applying the compositions for selective weed control in the presence of crops and should not react chemically with the compounds of the chemical formula herein or other composition ingredients. Such mixtures can be designed for application directly to weeds or their locus or can be concentrates or formulations which are normally diluted with additional carriers and adjuvants before application. They may include inert or active components and can be solids, such as, for example, dusts, granules, water dispersible granules, or wettable powders, or liquids, such as, for example, emulsifiable concentrates, solutions, emulsions or suspensions. Suitable agricultural carriers useful in preparing agricultural compositions of the present invention are well known to those skilled in the art. For example, liquid carriers that can be employed include water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, and the like. Water is generally the carrier of choice for the dilution of concentrates. Suitable solid carriers include talc, pyrophyllite clay, silica, attapulgus clay, kieselguhr, chalk, diatomaceous earth, lime, calcium carbonate, bentonire clay, Fuller's earth, cotton seed hulls, wheat flour, soybean flour, pumice, wood flour, walnut shell flour, lignin, and the like.

It is frequently desirable to incorporate one or more surface-active agents into the compositions of the present invention. Such surface-active agents are advantageously employed in both solid and liquid compositions, especially those designed to be diluted with carrier before application. The surface-active agents can be anionic, cationic or nonionic in character and can be employed as emulsifying agents, wetting agents, suspending agents, or for other purposes. Typical surface active agents include salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C.sub.18 ethoxylate; alcohol-alkylene oxide addition products, such as tridecyl alcohol-C.sub.16 ethorylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include antifoam agents, compatibilizing agents, sequestering agents, neutralizing agents and buffers, corrosion inhibitors, dyes, odorants, penetrations aids, spreading agents, sticking agents, dispersing agents, thickening agents, freeze point depressants, antimicrobial agents, and the like. The compositions can also contain other compatible components, for example, other herbicides, plant growth regulants, fungicides, insecticides, and the like and can be formulated with liquid fertilizers or solid, particulate fertilizer carriers such as ammonium nitrate, urea and the like.

“Agriculturally acceptable salt” is intended to mean a salt that retains the biological effectiveness of the free acids and bases of a specified compound and that is not biologically or otherwise undesirable. Examples of agriculturally acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, agricultural, biological, and biochemical arts.

As used herein the term “plant growth regulator” refers but not limited to a compound, either natural or synthetic, that modifies or controls one or more specific physiological processes within a plant.

As used herein the term “plant” refers but not limited to whole plants, ancestors and progeny of the plants and plant parts, including seeds, shoots, stems, roots (including tubers), and plant cells, tissues and organs. The plant may be in any form including suspension cultures, embryos, meristematic regions, callus tissue, leaves, gametophytes, sporophytes, pollen, and microspores.

As used herein the term “crop protection agent” refers but not limited to an agent which is a pesticide (or a mixture of more than one pesticide) or a plant growth regulator.

As used herein the term “pesticide” refers to, but not limited to a chemical or biological agent that deters, incapacitates, kills, or otherwise discourages pests.

Compounds of this invention will generally be used as a herbicidal active ingredient in a composition. As used herein the term “composition” refers but not limited to a formulation, with at least one additional component selected from the oup consisting of surfactants, solid diluents and liquid diluents, which serves as a carrier. The formulation or composition ingredients are selected to be consistent with the physical properties of the active ingredient, mode of application and environmental factors such as oil type, moisture and temperature. Useful compositions may include both liquid and solid formulation. Liquid formulations may include solutions (including emulsifiable concentrates), suspensions, emulsions (including icroemulsions, oil-in-water emulsions, flowable concentrates and/or suspoemulsions) and alike, which optionally can be thickened into gels. The general types of aqueous liquid compositions are soluble concentrate, suspension concentrate, capsule suspension, oncentrated emulsion, microemulsion, oil-in-water emulsion, flowable concentrate and uspo-emulsion. The general types of nonaqueous liquid compositions are emulsifiable oncentrate, microemulsifiable concentrate, dispersible concentrate and oil dispersion.

The general types of solid formulations are dusts, powders, granules, pellets, prills, astilles, tablets, filled films (including seed coatings) and the like, which can be ater-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively, the entire formulation of active ingredient can be encapsulated (or “overcoated”). Encapsulation can control or delay release of the active ingredient. Sprayable formulations are typically extended in a suitable medium before spraying. Such liquid and solid formulations are formulated to be readily diluted in the spray medium, usually water, but occasionally another suitable medium like an aromatic or paraffinic ydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by eight.

The compounds of the invention have (both preemergent and postemergent) herbicidal activity. As used herein the term “controlling undesired plant growth” refers to killing or injuring the vegetation or reducing its growth.

The compounds and compositions of the invention can be usefully applied by a variety of methods involving contacting a herbicidally effective amount of a compound of the invention, or a composition comprising said compound and at least one of a surfactant, a solid diluent or a liquid diluent, to the foliage or other part of the undesired plant or to the environment of the undesired plant growth such as the soil or water in which the undesired plant is growing or which surrounds the seed or other propagule of the undesired plant.

A herbicidally effective amount of the compounds of this invention is determined by a number of factors: formulation selected, method of application, amount and type of undesired plant growth present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

The compounds of the invention are applied, typically in a form of formulated composition, to a locus comprising desired vegetation (e.g., crops) and undesired plant growth (i.e. weeds), both of which may be seeds, seedlings and/or larger plants, in contact with a growth medium (e.g., soil). In this locus, a composition comprising the compounds of the invention can be directly applied to a plant or any part of a plant thereof, particularly of the undesired plant growth, and/or to the growth medium in contact with the plant.

Compounds of this invention can also be mixed with one or more other biologically active compounds or agents including herbicides, herbicide safeners, fungicides, insecticides, nematocides, bactericides, acaricides, plant growth regulators such as insect molting inhibitors and rooting stimulants, chemosterilants, semiochemicals, repellents, attractants, pheromones, feeding stimulants, plant nutrients, other biologically active compounds or bacteria, virus or fungi to form a multi-component pesticide giving broader spectrum of agricultural protection. Mixtures of the compounds of the invention with other herbicides can broaden the spectrum of activity against additional weed species and suppress the proliferation of any resistant biotypes.

Throughout this application various publications, published patent applications and published patents are referenced. The disclosures of these publications in their entireties are hereby incorporated by reference into this application in order to more fully describe the state of the art to which this invention pertains.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1-68. (canceled)

69. An agricultural composition comprising a Compound having the structure 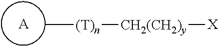
or a salt thereof, wherein:
A is five, six or nine-membered ring comprising from 1 to 3 heteroatoms; wherein each of the heteroatoms is independently selected from the group consisting of N, S, and O; and wherein one or more of the carbon atoms of the ring are optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein the 1-3 heteroatoms of the ring are N or S, each independently is optionally substituted with R₃, R₄, R₆ and R₇;
T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;
n is 0 or 1;
y is 0 to 8;
X is 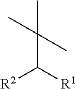
wherein R₁ is selected from the group consisting of carboxyl group, carbamoyl group, ester with saturated or non-saturated alcohols with straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide, and at least one agriculturally acceptable carrier.

70. The agricultural composition of claim 69, wherein A is selected from the group consisting of pyrrole, pyrazole, imidazole, triazole, thiophene, dithiole, trithiole, thiazole, dithiazole, trithiazolidine, thiadiazole, dithiadiazole, thiatriazole, oxazole, furan, oxathiole, pyridine, pyridazine, pyrimidine, pyrazine, triazine, thiazine, dithiazine, thiadiazine, 2H-1,3,4,5-thiatriazine, dithiadiazine, pyrrolopyridine, pyrrolopyrimidine, pyrrolotriazine, thiopyranopyrrole, pyrrolothiazine, pyrrolodithiazine, thienopyridine, thiophene, 1H-pyrazol, 2H-1,3-dithiole, 3H-1,2-dithiole, 1H-pyrrole, 2H-1,2,3-triazole, 1H-1,2,4-triazole, 1,3-thiazole, 1,2,5-thiadiazole, 1,2,4-thiadiazole, 1,3-oxazole, furan, and pyridine.

71. The agricultural composition of claim 69, wherein EWG is selected from the group consisting of triflyl (trifluoromethanesulfonyl), trihalide, cyano, sulfonate, nitro, ammonium, quaternary amine, aldehyde, ketone, carboxylic acid, ester, amide, and halide.

72. The agricultural composition of claim 69, wherein EDG is selected from the group consisting of phenoxide, tertiary amine, secondary amine, primary amine, ether, alkyl, phenyl and vinyl.

73. The agricultural composition of claim 69, wherein R₂ is selected from the group consisting of primary, secondary and tertiary amine substituted with a group selected from hydrogen, an aliphatic compound with straight or branched chain, an aliphatic compound with non-aromatic ring, straight or branched-chain alcohol, alcohol with non-aromatic ring, carboxylic acid, phosphoric acid, phosphonic acid, terminal carbon of phosphonate, ethylene glycol, polyethylene glycol, propylene glycol, polypropylene glycol, carboxamide with saturated or non-saturated straight, branched or cyclic chain, sulfonamide, and phosphoramide.

74. The agricultural composition of claim 69, comprising the compound having the structure selected from the group consisting of: 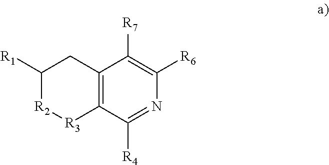
wherein R₁ is COOH, COOCH₃, COOC₂H₅, COOC₃H₇; R₂ is NH₂; R₃ and R₇ are independently selected from H, CF₃, CCl₃, Cl, and F; and R₄ and R₆ are independently selected from H, Cl, and Br; 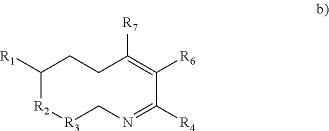
wherein R₁ is selected from COOH, COOCH₃, COOC₂H₅, and COOC₃H₇; R₂ is NH₂; R₃ and R₄ are independently selected from H, Br and Cl; and R₆ and R₇ are independently selected from H, Cl, NO₂, CF₃, and CCl₃; 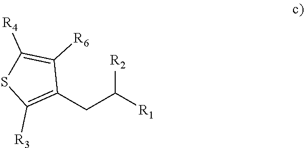
wherein R₁ is selected from COOH, COOCH₃, COOC₂H₅, COOC₄H₉, CONHCH₂COOH, and COO(CH₂)₃CH₃; R₂ is selected from NH₂, NHCOCH₃, NHCO, and NH(CH₂)₂O(CH₂)₂)OH; R₃ and R₄ are independently selected from H, Cl, Br, CN, CHF2 CF₃, CCl₃; and R₆ is H or CF₃; 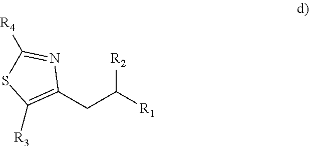
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₄ are each independently selected from H, Br, Cl, and NH₂; 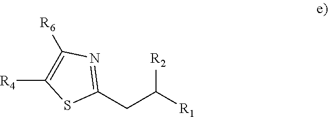
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₄ and R₆ are each independently selected from H, Br, Cl, CF₃, CHF₂, and CCl₃; 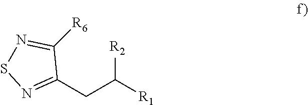
wherein R₁ is selected from the group consisting of COOH; R₂ is NH₂; and R₆ is H; 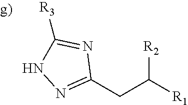
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂; 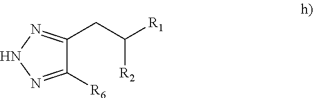
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂; and, 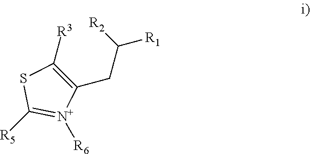
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₆ is selected from CH₃, OH and NH₂.

75. The agricultural composition of claim 69, further comprising at least one crop protection agent.

76. The agricultural composition of claim 75, wherein the at least one crop protection agent is selected from the group consisting of fungicide, insecticide, herbicide, and plant growth regulator.

77. The agricultural composition of claim 75, wherein the at least one crop protection agent is selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop(P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof.

78. The agricultural composition of claim 69, comprising the compound selected from the group consisting of 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2.5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride 3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate, or an agriculturally acceptable salt thereof.

79. A method of controlling undesired plant growth comprising applying to the locus of said undesired plant growth a herbicidally effective amount of a compound having the structure 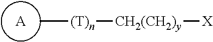
or a salt thereof, wherein:
A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;
T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;
n is 0 or 1;
y is 0 to 8;
X is 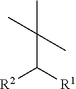
wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide.

80. A method of controlling undesired plant growth, comprising applying to the locus of the undesired plant growth an agricultural composition of claim 69.

81. The method of claim 80,
wherein A is selected from the group consisting of pyrrole, pyrazole, imidazole, triazole, thiophene, dithiole, trithiole, thiazole, dithiazole, trithiazolidine, thiadiazole, dithiadiazole, thiatriazole, oxazole, furan, oxathiole, pyridine, pyridazine, pyrimidine, pyrazine, triazine, thiazine, dithiazine, thiadiazine, 2H-1,3,4,5-thiatriazine, dithiadiazine, pyrrolopyridine, pyrrolopyrimidine, pyrrolotriazine, thiopyranopyrrole, pyrrolothiazine, pyrrolodithiazine, thienopyridine, thiophene, 2H-1,3-dithiole, 3H-1,2-dithiole, 1H-pyrrole, 2H-1,2,3-triazole, 1H-1,2,4-triazole, 1,3-thiazole, 1,2,5-thiadiazole, 1,2,4-thiadiazole, 1,3-oxazole, furan, and pyridine;
wherein EWG is selected from the group consisting of triflyl (trifluoromethanesulfonyl), trihalide, cyano, sulfonate, nitro, ammonium, quaternary amine, aldehyde, ketone, carboxylic acid, ester, amide, and halide;
wherein EDG is selected from the group consisting of phenoxide, tertiary amine, secondary amine, primary amine, ether, alkyl, phenyl and vinyl; and/or
wherein R₂ is selected from the group consisting of primary, secondary and tertiary amine substituted with a group selected from hydrogen, an aliphatic compound with straight or branched chain, an aliphatic compound with non-aromatic ring, straight or branched-chain alcohol, alcohol with non-aromatic ring, phosphoric acid, phosphonic acid, terminal carbon of phosphonate, ethylene glycol, polyethylene glycol, propylene glycol, polypropylene glycol, carboxamide with saturated or non-saturated straight, branched or cyclic chain, sulfonamide, and phosphoramide.

82. The method of claim 80 comprising applying to the locus of the undesired plant growth an agricultural composition comprising the compound having the structure selected from the group consisting of: 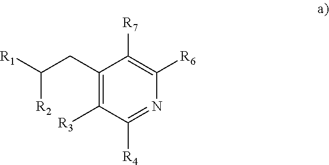
wherein R₁ is COOH, COOCH₃, COOC₂H₅, COOC₃H₇; R₂ is NH₂; R₃ and R₇ are independently selected from H, CF₃, CCl₃, Cl, and F; and R₄ and R₆ are independently selected from H, Cl, and Br;
b) 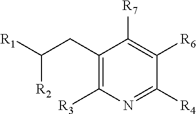
wherein R₁ is selected from COOH, COOCH₃, COOC₂H₅, and COOC₃H₇; R₂ is NH₂; R₃ and R₄ are independently selected from H, Br and Cl; and R₆ and R₇ are independently selected from H, Cl, NO₂, CF₃, and CCl₃; 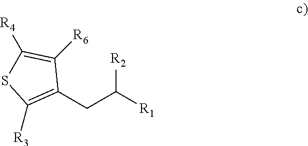
wherein R₁ is selected from COOH, COOCH₃, COOC₂H₅, COOC₄H₉, CONHCH₂COOH, and COO(CH₂)₃CH₃; R₂ is selected from NH₂, NHCOCH₃, NHCO, and NH(CH₂)₂O(CH₂)₂)OH; R₃ and R₄ are independently selected from H, Cl, Br, CN, CHF2 CF₃, CCl₃; and R₆ is H or CF₃; 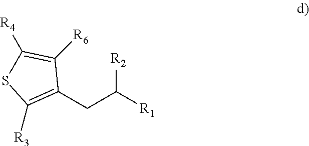
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₄ are each independently selected from H, Br, Cl, and NH₂; 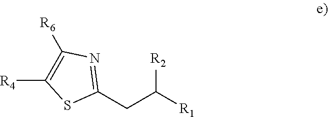
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₄ and R₆ are each independently selected from H, Br, Cl, CF₃, CHF₂, and CCl₃; 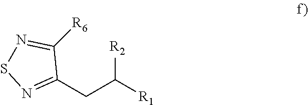
wherein R₁ is COOH; R₂ is NH₂; and R₆ is H; 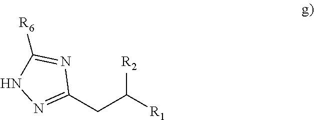
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂; 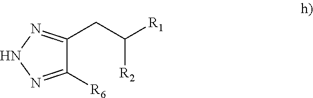
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; and R₆ is selected from H, Br, Cl, CF₃, CCl₃, and NH₂; and 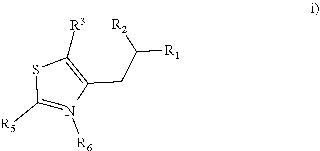
wherein R₁ is selected from COOH, COOCH₃, COOC₂H₅, COOC₅H₁₁, and COO(CH₂)₂(CH₂)₂CH₃; R₂ is selected from NH₂, NHCOCH₃, or NHCOC₂H₅; R₃ and R₅ are independently selected from H, Br, Cl, CF₃, CCl₃; and R₆ is selected from CH₃, OH and NH₂.

83. The method of claim 80, further comprising applying to the locus of the undesired plant growth at least one crop protection agent,
wherein the at least one crop protection agent is amino acid synthesis inhibitor herbicide selected from the group consisting of sulfonylurea herbicide, imidazolinone herbicide, sulfonamide herbicide, amino acid derivatives, imazamox, imazapic, imazethapyr, imazaquin, imazapyr and imazamethabenz, Chlorimuron, Primisulfuron, Thifensulfuron, Triasulfuron, Nicosulfuron, Metsulfuron, Tribenuron, Rimsulfuron, Triflusulfuron, and glyphosate; and/or
wherein the at least one crop protection agent is a plant growth regulator selected from the group consisting of dicamba, 2,4-D, clopyralid and fluroxypyr.

84. A method of controlling undesired plant growth comprising applying to the locus of the undesired plant growth:
a. a first herbicide having the structure 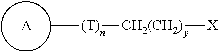
or a salt thereof, wherein:
A is five, six or nine-membered ring comprising one or more heteroatoms; wherein each of the heteroatoms is independently selected from the group consisting of N, S, and O; and wherein one or more of the carbon atoms of the ring is optionally substituted with R₃, R₄, R₆ and R₇; and wherein each of R₃, R₄, R₆ or R₇ is independently selected from and an electron withdrawing group (EWG) or an electron donating group (EDG); wherein when R₃, R₄, R₆ or R₇ is EDG, at least one carbon of the ring is substituted with EWG; and wherein, when one or more heteroatoms of the ring is N or S, each independently is optionally substituted with R₃, R₄, R₆ and R₇;
T is selected from the group consisting of sulfonyl, ammonium, alkylamine; arylamine, phosphonium, nitro, cyano, dihalomethyl, acyl, formyl, haloformyls, carboxyl, alkoxycarbonyl, and aminocarbonyl;
n is 0 or 1;
y is 0 to 8;
X is 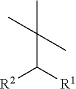
wherein R₁ is selected from the group consisting of carboxyl group, carbamoyl group, ester with saturated or non-saturated alcohols straight, branched, cyclic chain or aromatic chain, ethylene glycol, polyethylene glycol, propylene glycol, polypropylene glycol, carboxamide substituted with straight, branched, cyclic aliphatic chain, cyclic aromatic chain, ethylene glycol, polyethylene glycol, propylene glycol, polypropylene glycol, sulfonamide, and phosphoramide; and wherein R₂ is selected from the group consisting of optionally substituted primary amine, secondary amine, tertiary amine, carboxamide, sulfonamide, and phosphoramide; and
b. a second herbicide, to effectively control the undesired plant growth.

85. The method of claim 84, wherein the second herbicide is amino acid synthesis inhibitor herbicide selected from the group consisting of sulfonylurea herbicide, imidazolinone herbicide, sulfonamide herbicide, amino acid derivatives, imazamox, imazapic, imazethapyr, imazaquin, imazapyr and imazamethabenz, Chlorimuron, Primisulfuron, Thifensulfuron, Triasulfuron, Nicosulfuron, Metsulfuron, Tribenuron, Rimsulfuron, Triflusulfuron, and glyphosate.

86. The method of claim 84, wherein the second herbicide is selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop(P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, and ethametsulfuron-methyl.

87. The method of claim 84, further comprising applying a third herbicide or a plant growth regulator.

88. The method of claim 79, comprising applying to the locus of the undesired plant growth a compound or a salt thereof, selected from the group consisting of: 2-amino-3-(thiophen-2-yl)propanoic acid; 2-amino-3-(thiophen-3-yl)propanoic acid; 2-amino-3-[(thiophene-3-sulfonyl)amino]propanoic acid; 2-amino-3-[(4-fluorothiophene-3-sulfonyl)amino]propanoic acid; methyl 2-amino-3-[(thiophene-3-sulfonyl)amino]propanoate; pentyl 2-amino-3-[(thiophene-3-sulfonyl)amino]propanoate; 3-(5-cyano-3-thienyl)alanine; 2-amino-3-[4-(trifluoromethyl)thiophen-2-yl]propanoic acid; 3-amino-3-(4-bromothiophen-3-yl)propanoic acid; 2-amino-3-(2,4,5-trimethylthiophen-3-yl)propanoic acid; butyl 2-amino-3-(thiophen-3-yl)propanoate; 2-amino-3-[(2,5-dichlorothiophene-3-sulfonyl)amino]propanoic acid; 2-amino-3-[(2,5-dichloro-4-methylthiophene-3-sulfonyl)amino]propanoic acid; 2-amino-3-(2-chlorothiophen-3-yl)propanoic acid; 2-amino-3-(2,5-dichlorothiophen-3-yl)propanoic acid; 2-amino-3-(5-chlorothiophen-3-yl)propanoic acid; 2-amino-3-(2,5-dihydro-1,3-thiazol-4-yl)propanoic acid; 2-amino-3-(1,3-thiazol-5-yl)propanoic acid; 2-amino-3-{[2,5-dichloro-4-(trifluoromethyl)thiophene-3-sulfonyl]amino}propanoic acid; 2-amino-3-(2-amino-1,3-thiazol-4-yl)propanoic acid; methyl 2-amino-3-[(2,5-dichlorothiophene-3-sulfonyl)amino]propanoate; ethyl 2-amino-3-[(2,5-dichlorothiophene-3-sulfonyl)amino]propanoate; 2-propoxyethyl-2-amino-3-[(2,5-dichlorothiophene-3-sulfonyl)amino]propanoate; [2-(carboxyamino)-3-ethoxy-3-oxopropyl](2,5-dichlorothiophene-3-sulfonyl)carbamic acid; 2-amino-3-(2,5-dichloro-1,3-thiazol-4-yl)propanoic acid; 2-amino-3-(5-chloro-1,3-thiazol-4-yl)propanoic acid; 2-amino-3-(1,2,5-thiadiazol-3-yl)propanoic acid; 2-amino-3-(1,2,4-thiadiazol-5-yl)propanoic acid; 2-amino-3-(furan-3-yl)propanoic acid; 2-amino-3-(1,3-oxazol-4-yl)propanoic acid; 2-formamido-3-(thiophen-3-yl)propanoic acid; methyl 2-amino-3-(thiophen-3-yl)propanoate; ethyl 2-amino-3-(thiophen-3-yl)propanoate; [2-amino-3-(thiophen-3-yl)propanamido]acetic acid; 2-amino-N-[2-(2-hydroxyethoxy)ethyl]-3-(thiophen-3-yl)propenamide; ethyl 2-butanamido-3-(dichloro-1,3-thiazol-4-yl)propanoate; 2-butoxyethyl 2-acetamido-3-(2,5-dichloro-1,3-thiazol-4-yl)propanoate; 2-amino-3-(2,5-dibromothiophen-3-yl)propanoic acid; 2-amino-3-(5-nitrothiophen-3-yl)propanoic acid; butyl 3-(dichloro-1,3-thiazol-4-yl)-2-acetamidopropanoate; 2-ethoxyethyl 3-(dichloro-1,3-thiazol-4-yl)-2-acetamidopropanoate; 2-amino-3-[5-(trifluoromethyl)thiophen-3-yl]propanoic acid; (2S)-2-amino-3-(3,5-dioxo-1,2,4-oxadiazolidin-2-yl)propanoic acid; 2-amino-3-[(2,5-dibromothiophene-3-sulfonyl)amino]propanoic acid; 2-amino-3-(5-chloro-2-methylthiophen-3-yl)propanoic acid; 2-amino-3-(2-bromothiophen-3-yl)propanoic acid; 2-amino-3-(1,1-dioxo-116-thiolan-3-yl)propanoic acid; 2-amino-3-(thiolan-3-yl)propanoic acid; [2-amino-2-(thiophen-3-yl)ethyl]phosphonic acid; 2-amino-3-(2,5-dimethyl-1,1-dioxo-1H-116-thiophen-3-yl)propanoic acid; 3-(2,5-dioxo-2,5-dihydrothiophen-3-yl)-2-(phosphonoamino)propanoic acid; 2-amino-3-[4-(trifluoromethyl)thiophen-3-yl]propanoic acid; ethyl 2-acetamido-3-(2,5-dichloro-1,3-thiazol-4-yl)propanoate; methyl 2-amino-3-(2,5-dichlorothiophen-3-yl)propanoate; ethyl 2-amino-3-(2,5-dichlorothiophen-3-yl)propanoate; 2-(2-methoxyethoxy)ethyl-2-amino-3-(thiophen-3-yl)propanoate; ethyl 2-acetamido-3-(thiophen-3-yl)propanoate; ethyl 3-(2,5-dichlorothiophen-3-yl)-2-acetamidopropanoate; ethyl 3-(2,5-dichlorofuran-3-yl)-2-acetamidopropanoate; propyl 2-amino-3-(2,5-dichlorofuran-3-yl)propanoate; 4-(2-acetamido-3-ethoxy-3-oxopropyl)-3-methyl-1,3-thiazol-3-ium chloride; 2-(2-ethoxyethoxy)ethyl 3-(2,5-dichlorothiophen-3-yl)-2-acetamidopropanoate; pentyl 2-acetamido-3-(1,3,4-oxadiazol-2-yl)propanoate; ethyl-2-amino-3-[(2,5-dibromothiophene-3-sulfonyl)amino]propanoate; butyl 3-(5-chloro-1,2,4-thiadiazol-3-yl)-2-acetamidopropanoate; 2-amino-3-(1H-indazol-3-yl)propanoic acid hydrochloride[HCl]; 2-amino-3-(pyridazin-4-yl)propanoic acid; 2-amino-3-(pyridin-4-yl)propanoic acid; 2-amino-3-(pyridin-3-yl)propanoic acid dihydrochloride
3-(5-Hydroxy-3-pyridinyl)alanine; 2-amino-3-(5-methylpyridin-3-yl)propanoic acid; 2-amino-3-(4-aminopyridin-2-yl)propanoic acid; 2-amino-3-(2-aminopyridin-4-yl)propanoic acid; 2-amino-3-(6-aminopyridin-2-yl)propanoic acid; 2-amino-3-(5-aminopyridin-3-yl)propanoic acid; 2-amino-3-(5-sulfanylpyridin-3-yl)propanoic acid; 2-amino-3-(2-sulfanylpyridin-4-yl)propanoic acid; 2-amino-3-(pyrimidin-5-yl)propanoic acid; 2-amino-3-(4-amino-6-chloropyrimidin-2-yl)propanoic acid; 2-amino-3-(pyrimidin-2-yl)propanoic acid dihydrochloride; 2-amino-3-(4-hydroxypyrimidin-2-yl)propanoic acid; 2-amino-3-(2,4-dioxo-1,2,3,4-tetrahydropyrimidin-5-yl)propanoic acid; 2-amino-3-(pyrazin-2-yl)propanoic acid; 2-amino-3-(5-chloropyridin-3-yl)propanoic acid; 2-amino-3-(2,5-dichloropyridin-3-yl)propanoic acid; 2-amino-3-(pyrimidin-4-yl)propanoic acid; 2-amino-3-(5-fluoropyridin-3-yl)propanoic acid; 2-amino-3-(6-chloropyridin-3-yl)propanoic acid; 2-amino-3-(2,6-dichloropyridin-4-yl)propanoic acid; 2-amino-3-(3,5-dichloropyridin-4-yl)propanoic acid; 2-amino-3-(3,5-difluoropyridin-4-yl)propanoic acid; 2-amino-3-[2-(dimethylamino)pyridin-4-yl]propanoic acid; 2-amino-3-(2-bromopyridin-4-yl)propanoic acid; 2-amino-3-(5,6-dibromopyridin-3-yl)propanoic acid; 2-amino-3-(2,6-dibromopyridin-4-yl)propanoic acid; 2-amino-3-(2,3-dibromopyridin-4-yl)propanoic acid; 2-amino-3-[5-(trifluoromethyl)pyridin-3-yl]propanoic acid; 2-amino-3-(5,6-dichloropyridin-3-yl)propanoic acid; 2-amino-3-(2-oxo-1,2-dihydropyridin-4-yl)propanoic acid; 2-amino-3-[4-(dimethylamino)pyridin-3-yl]propanoic acid; 2-amino-3-(2-oxo-1,2-dihydropyridin-3-yl)propanoic acid; 2-amino-3-(6-oxo-1,6-dihydropyrimidin-4-yl)propanoic acid; 2-amino-3-(6-oxo-1,6-dihydropyrimidin-5-yl)propanoic acid; 2-amino-3-(6-oxo-1,6-dihydropyridin-3-yl)propanoic acid; 2-amino-3-(2,6-dioxo-1,2,3,6-tetrahydropyrimidin-4-yl)propanoic acid; 2-amino-3-(6-oxo-2-sulfanyl-1,6-dihydropyrimidin-4-yl)propanoic acid; propyl 2-amino-3-(2-bromopyridin-4-yl)propanoate; 2-amino-3-(2-bromo-3,5-difluoropyridin-4-yl)propanoic acid; propyl 2-amino-3-(2-bromo-3,5-difluoropyridin-4-yl)propanoate; 2-amino-3-[(2-bromopyridine-4-sulfonyl)amino]propanoic acid; propyl 2-amino-3-[(2-bromopyridine-4-sulfonyl)amino]propanoate; ethyl 2-amino-3-(5,6-dibromopyridin-3-yl)propanoate; ethyl 2-amino-3-(2,3-dibromopyridin-4-yl)propanoate acid; 2-amino-3-[2-chloro-5-(trifluoromethyl)pyridin-3-yl]propanoic acid; 2-amino-3-(2-amino-6-oxo-1,6-dihydropyrimidin-5-yl)propanoic acid; 2-amino-3-(6-hydroxy-5-nitropyridin-3-yl)propanoic acid; methyl 2-amino-3-(2-bromopyridin-4-yl)propanoate; 3-(2-amino-2-carboxyethyl)-1-methylpyridin-1-ium; 3-(2-amino-2-carboxyethyl)-1-hydroxypyridin-1-ium; 2-amino-3-(4-amino-6-hydroxy-2-methylpyrimidin-5-yl)propanoic acid; methyl 2-amino-3-(2,6-dibromopyridin-4-yl)propanoate; ethyl 3-(6-bromopyridin-3-yl)-2-acetamidopropanoate; methyl 3-(6-bromopyridin-3-yl)-2-acetamidopropanoate; ethyl-2-acetamido-3-(2-methoxypyrimidin-4-yl)propanoate; 2-(2-methoxyethoxy)ethyl 2-amino-3-(6-bromopyridin-3-yl)propanoate; 2-(2-ethoxyethoxy)ethyl 2-amino-3-(6-bromopyridin-3-yl)propanoate; 5-(2-amino-2-carboxyethyl)-1-methylpyrimidin-1-ium; 2-amino-3-[2-(trifluoromethyl)pyridin-4-yl]propanoic acid hydrochloride[HCl]; ethyl 3-[(2-bromopyridin-4-yl)oxy]-2-acetamidopropanoate; ethyl 2-acetamido-3-(pyridin-4-yloxy)propanoate; 2-amino-4-(1H-pyrrol-3-yl)butanoic acid; 2-amino-3-(1H-pyrrol-2-yl)propanoic acid; 2-amino-3-(1H-pyrrol-3-yl)propanoic acid; 2-amino-3-(4H-1,2,4-triazol-3-yl)propanoic acid; (2S)-2-hydroxy-3-(1H-imidazol-4-yl)propanoic acid; ethyl 2-{[(benzyloxy)carbonyl]amino}-3-(1H-1,2,4-triazol-3-yl)propanoate; 2-amino-3-(4-methyl-4H-1,2,4-triazol-3-yl)propanoic acid; 2-amino-3-(1-methyl-1H-1,2,4-triazol-5-yl)propanoic acid dihydrochloride; 2-amino-3-(1-methyl-1H-1,2,3-triazol-4-yl)propanoic acid hydrochloride; 2-amino-3-(2-methyl-2H-1,2,3,4-tetrazol-5-yl)propanoic acid hydrochloride; 2-amino-3-(dimethyl-4H-1,2,4-triazol-3-yl)propanoic acid hydrochloride; (2S)-2-amino-3-[5-(trifluoromethyl)-1H-pyrazol-4-yl]propanoic acid; and 2-(2-butoxyethoxy)ethyl 2-amino-3-(2H-1,2,3-triazol-4-yl)propanoate.
